(19) United States
(12) Patent Application Publication
(10) Pub. No.: US 2003/0180747 A1
(43) Pub. Date: Sep. 25, 2003

(54) Pancreatic cancer diagnosis and therapies

(76) Inventors: Ralph H. Hruban, Baltimore, MD (US); Pedram Argani, Pikesville, MD (US); Christine A. Iacobuzio-Donahue, Columbia, MD (US); Anirban Maitra, Baltimore, MD (US)

Correspondence Address: EDWARDS & ANGELL, LLP, P.O. BOX 9169, BOSTON, MA, 02209, US

(21) Appl. No.: 10/269,909

(22) Filed: Oct. 11, 2002

Related U.S. Application Data
(60) Provisional application No. 60/328,609, filed on Oct. 11, 2001.
(60) Provisional application No. 60/332,754, filed on Nov. 19, 2001.

(51) Int. Cl. C12Q 1/68; A61K 48/00

(52) U.S. Cl. 435/6; 514/44; 435/320.1

(57) ABSTRACT

The present invention includes methods and systems for identification (diagnosis) of abnormal cell growth by analysis of a patient sample, particularly the presence of a pancreatic cancer or susceptibility to a pancreatic cancer. The invention also includes therapeutic agents for treating pancreatic cancers as well as methods for identifying candidate agents for treatment of pancreatic cancers.

The present application claims the benefit of U.S. provisional application No. 60/328,609, filed Oct. 11, 2001; U.S. provisional application No. 60,332,754, filed Nov. 19, 2001; all of which are incorporated by reference herein in their entirety.

### BACKGROUND OF THE INVENTION

1. Field of the Invention

The present invention includes methods and systems for identification (diagnosis) of abnormal cell growth by analysis of a patient sample, particularly the presence of a pancreatic cancer or susceptibility to a pancreatic cancer. The invention also includes therapeutic agents for treating pancreatic cancers as well as methods for identifying candidate agents for treatment of pancreatic cancers.

2. Background

Pancreatic cancer continues to have one of the highest mortality rates of any malignancy. Each year, 28,000 patients are diagnosed with pancreatic cancer, and most will die of the disease. The vast majority of patients are diagnosed at an advanced stage of disease because currently no tumor markers are known that allow reliable screening for pancreas cancer at an earlier, potentially curative stage. This is a particular problem for those patients with a strong familial history of pancreatic cancer, who may have up to a 5-7 fold greater risk of developing pancreatic cancer in their lifetime. Despite several advances in our basic understanding and clinical management of pancreatic cancer, virtually all patients who will be diagnosed with pancreatic cancer will die from this disease. The high mortality of pancreatic cancer is predominantly due to consistent diagnosis at an advanced stage of disease, and a lack of effective screening methods.

There is a an urgent need, therefore, for new systems for detection and diagnosis of pancreatic cancers, particularly at a pre-invasive or early stage of the disease so that early medical intervention can be more effective at saving lives.

### SUMMARY OF THE INVENTION

The invention provides methods for the detection of pancreatic cancer at an early stage of the disease that can allow for early medical treatment and enhanced patient survival rates.

In particular, the invention provides methods for the identification of upregulated gene expression and variants thereof, which are not known to be expressed in the pancreas. Large numbers of genes differentially expressed in a large series of pancreatic cancers are identified using biochip arrays. Biocomputational tools are utilized to determine those genes most highly expressed within pancreatic cancer samples compared to normal pancreatic tissue.

In a preferred embodiment for detection of cancer, high-through put screening assays, such as the Gene Logic Inc. BioExpress™ platform and Affymetrix GeneChip® arrays, are utilized for screening of thousands of genes, fragments or variants thereof. Different types of pancreatic cancers are preferably detected by the identification of one or more nucleic acid molecules in a subject sample, wherein at least one of the molecules comprises a sequence corresponding to a molecule identified in any of FIGS. 1A through 1M. Preferably, genes found to be significantly expressed in SAGE libraries of normal pancreatic ductal cells are excluded and the expression of selected genes are confirmed by immunohistochemical labeling, in situ hybridization and RT-PCR.

The presence of the one or more nucleic acid molecules is correlated to a sample of a normal subject. The sample is preferably obtained from a mammal suspected of having a proliferative cell growth disorder, in particular, a pancreatic cancer.

In a preferred embodiment a nucleic acid molecule that is indicative of a pancreatic cancer comprises a sequence having at least about 80% sequence identity to a molecule identified in any of FIGS. 1A through 1M, more preferably the nucleic acid molecule comprises a sequence having at least about 90% sequence identity to a molecule identified in any of FIGS. 1A through 1M, most preferably the nucleic acid molecule comprises a sequence having at least about 95% sequence identity to a molecule identified in any of FIGS. 1A through 1M.

In another preferred embodiment, the nucleic acid molecule is expressed at a higher level in a patient with cancer as compared to expression levels in a normal individual. Preferably the nucleic acid molecule is expressed at least about 10 fold higher in a patient with cancer as compared to expression in a normal individual, more preferably the nucleic acid molecule is expressed at least about 10 fold higher in a patient with cancer as compared to expression in a normal individual, most preferably the nucleic acid molecule is expressed at least about 5 fold higher in a patient with cancer as compared to expression in a normal individual.

In other preferred embodiments the nucleic acid molecule encodes for a membrane bound protein, cytoplasmic protein, nuclear protein or extracellular protein. Examples of such proteins, include but are not limited to OB-cadherin, fascin, ATDC, topoisomerase II alpha, pleckstrin, paraneoplastic antigen MA1, heat shock protein 47, thrombospondin 2, or osteopontin.

In another preferred embodiment, the sample used for detection of preferred nucleic acid molecules is obtained from a mammalian patient, including a human patient.

In another preferred embodiment, a polypeptide encoded by the nucleic acid molecule is detected. The polypeptide is preferably detected in a subject sample, suffering from or susceptible to, any type of pancreatic cancer and the polypeptide is encoded by a sequence comprising a sequence identified in any of FIGS. 1A through 1M. Preferably the polypeptide is encoded by a sequence having at least about 80% sequence identity to a molecule identified in any of FIGS. 1A through 1M more preferably the polypeptide is encoded by a sequence having at least about 90% sequence identity to a molecule identified in any of FIGS. 1A through 1M, most preferred, the polypeptide is encoded by a sequence having at least about 95% sequence identity to a molecule identified in any of FIGS. 1A through 1M. Preferably one or more nucleic acid molecules are used in combination with a known marker such as for example, prostate stem cell antigen.

In a preferred embodiment the polypeptide is expressed at least a higher level in a patient with cancer as compared to expression levels in normal individuals, preferably the polypeptide is expressed at least about 5 to about 10 fold higher in a patient with cancer as compared to expression in a normal individual. Preferably the cancer is a pancreatic cancer and the subject sample is obtained from a mammalian patient, including a human patient.

In another preferred embodiments, any candidate therapeutic agents are identified. A preferred method for identifying a candidate therapeutic agent comprises contacting a candidate agent with a nucleic acid molecule or expression product thereof where the nucleic acid molecule comprises a sequence identified in any of FIGS. 1A through 1M; and detecting interaction between the candidate agent and the nucleic acid molecule or expression product. Preferably the method identifies candidate therapeutic agents which interact with a nucleic acid molecule comprising a sequence having at least about 80% sequence identity to a sequence identified in any of FIGS. 1A through 1M, more preferably the nucleic acid molecule comprises a sequence having at least about 90% sequence identity to a sequence identified in any of FIGS. 1A through 1M, most preferably, the nucleic acid molecule comprises a sequence having at least about 95% sequence identity to a sequence identified in any of FIGS. 1A through 1M.

In accordance with the invention, a candidate therapeutic agent contacts a nucleic acid molecule or expression product thereof where the nucleic acid molecule or expression product are overexpressed in a mammal suffering from a pancreatic cancer, the interaction between the candidate agent and the nucleic acid molecule or expression product is detected. The candidate compound is selected from the group consisting of a protein, a peptide, an oligopeptide, a nucleic acid, a small organic molecule, a polysaccharide and a polynucleotide.

In a preferred embodiment the nucleic acid molecule or expression product are provided on a solid support wherein binding of the candidate compound is detected.

The invention also provides methods for treating a mammal suffering from cancer comprising administering to the mammal an antibody specific for a nucleic acid molecule or expression product thereof where the nucleic acid molecule comprises a sequence corresponding to a molecule identified in any of FIGS. 1A through 1M. In accordance with the invention, another method for treating a mammal suffering from cancer comprises administering a vector comprising at least one or more nucleic acid sequences identified in FIGS. 1A through 1M. Preferably the nucleic acid molecule comprises a sequence having at least about 80% sequence identity to a sequence identified in any of FIGS. 1A through 1M, more preferably the nucleic acid molecule comprises a sequence having at least about 90% sequence identity to a sequence identified in any of FIGS. 1A through 1M, most preferably the nucleic acid molecule comprises a sequence having at least about 95% sequence identity to a sequence identified in any of FIGS. 1A through 1M. The method can be used to treat a patient is suffering from a pancreatic cancer.

Pharmaceutical compositions are also provided, comprising an antibody or specific for a nucleic acid molecule or expression product thereof, or vector comprising at least one or more nucleic acid molecules, where the nucleic acid molecule comprises a sequence corresponding to a molecule identified in any of FIGS. 1A through 1M. Preferably the nucleic acid molecule comprises a sequence having at least about 80% sequence identity to a molecule identified in any of FIGS. 1A through 1M, more preferably the nucleic acid molecule comprises a sequence having at least about 90% sequence identity to a molecule identified in any of FIGS. 1A through 1M, most preferably the nucleic acid molecule comprises a sequence having at least about 95% sequence identity to a molecule identified in any of FIGS. 1A through 1M.

In accordance with the invention, an antibody specific for a nucleic acid molecule or expression product thereof, or vector comprising at least one or more nucleic acid sequence molecules, where the nucleic acid molecule comprises a sequence corresponding to a molecule identified in any of FIGS. 1A through 1M, is generated. The antibody is preferably specific for nucleic acid molecules comprising a sequence having at least about 80% sequence identity to a molecule identified in any of FIGS. 1A through 1M, more preferable the antibody is specific for nucleic acid molecules comprising a sequence having at least about 90% sequence identity to a molecule identified in any of FIGS. 1A through 1M, most preferable the antibody is specific for nucleic acid molecules comprising a sequence having at least about 95% sequence identity to a molecule identified in any of FIGS. 1A through 1M.

In accordance with the invention a vector comprising one or more nucleic acid molecules, or variants thereof, where the nucleic acid molecule comprises a sequence corresponding to a molecule identified in any of FIGS. 1A through 1M. The vector preferably comprises nucleic acid molecules comprising a sequence having at least about 80% sequence identity to a molecule identified in any of FIGS. 1A through 1M, more preferable the vector preferably comprises nucleic acid molecules comprising a sequence having at least about 90% sequence identity to a molecule identified in any of FIGS. 1A through 1M, most preferable more preferable the vector preferably comprises nucleic acid molecules comprising a sequence having at least about 95% sequence identity to a molecule identified in any of FIGS. 1A through 1M. Preferably, the vector generates an in vivo immune response resulting in the cytolysis of a cell overexpressing the product of the nucleic acid molecules.

Diagnostic kits are also provided comprising a reaction body and a molecule substantially complementary to a sequence corresponding to a molecule identified in any of FIGS. 1A through 1M. Preferably, the kit comprises a molecule comprising a sequence having at least about 80% sequence identity to a molecule identified in any of FIGS. 1A through 1M, more preferable at least about 90% sequence identity to a molecule identified in any of FIGS. 1A through 1M, most preferable the kit comprises a molecule comprising a sequence having at least about 95% sequence identity to a molecule identified in any of FIGS. 1A through 1M.

Preferably, the kit comprises written instructions for use of the kit for detection of cancer and the instructions provide for detecting one or more nucleic acid molecules.

Other aspects of the invention are disclosed infra.

### BRIEF DESCRIPTION OF THE DRAWINGS

FIGS. 1A through to 1M are tabulated results showing the highly expressed genes identified in pancreatic cell lines and tissues. In FIGS. 1A to 1C, the first column identifies the nucleic acid sequence fragment name (Affymetrix gene fragments identified by the GeneExpress platform); the second column identifies the known gene name; the third column identifies the Unigene accession number; the third column identifies the fold change in gene expression as compared to normal cell gene expression; the fourth column denotes the P values; the fifth column indicates eNorthern pattern (described in FIG. 2 below); the sixth column identifies the SAGE normal tags; the seventh column indicates the novel nucleic acid molecules identified and includes previously reported genes; the eighth column provides the cellular localization of the molecules.

In FIGS. 1D to 1M, the first column identifies the nucleic acid sequence fragment name (Affymetrix gene fragments identified by the GeneExpress platform); the second column identifies the known gene name; the third column identifies the Unigene accession number; the third column identifies the fold change in gene expression as compared to normal cell gene expression; the fourth column denotes the P values.

FIGS. 2A and 2B show the results of an eNorthern analysis of highly expressed Affymetrix gene fragments identified by the GeneExpress platform. FIG. 2A shows the results from an Affymetrix fragment for sea urchin fascin homolog, highly expressed in both pancreas cancer cell lines and tumor tissues compared to normal (“A pattern”). FIG. 2B shows the results from an Affymetrix fragment for heat shock protein 47, specifically overexpressed in pancreas cancer tumor tissues but not pancreas cancer cell lines or normal tissues (“B pattern”).

FIG. 3A-3D shows the results of gene expression by immunohistochemical and in situ hybridization in pancreatic cancers. FIG. 3A: Fascin. Strong cytoplasmic immunolabeling is noted within the infiltrating neoplastic epithelium, in contrast to normal pancreatic duct epithelium which is negative. FIG. 3B: Topoisomerase 11 alpha. Strong nuclear immunolabeling is noted within the neoplastic epithelium, in contrast to the normal pancreatic duct epithelium (black arrows) and desmoplastic stroma which are negative. FIG. 3C: Heat shock protein 47. Strong immunolabeling is noted of the desmoplastic stroma of the tumor, in contrast to the neoplastic epithelium which is negative. FIG. 3D: Pleckstrin. mRNA expression is detected within the neoplastic epithelium by in situ hybridization (black arrows), in contrast to the surrounding desmoplastic stroma which is negative.

FIG. 4 is a gel showing gene expression by RT-PCR.

### DETAILED DESCRIPTION OF THE INVENTION

The invention provides methods for simultaneously identifying novel tumor markers that are diagnostic of pancreatic cancers at the early and treatable stages of the disease, thus increasing survival rates in patients with pancreatic cancer. In particular, biochip arrays can be employed to identify genes differentially expressed in pancreatic cancer. Simultaneous analysis of 60,000 fragments, with 12,000 fragments covering full length genes and 48,000 fragments covering ESTs, genes expressed at least 5 fold greater in the pancreatic cancers compared to normal tissues were identified. Selected candidate genes are validated by immunohistochemical analysis, by in situ hybridization and RT-PCR. At least about 69 genes which had not been associated with pancreatic cancer before were identified. These molecules have immediate potential as novel therapeutic targets and tumor markers of pancreatic cancer.

In an aspect of the invention, it is shown that high-throughput gene profiling combined with effective use of bioinformatics tools offers a viable approach to screening for tumor markers. In particular, potential markers of pancreatic carcinoma are identified by utilizing the Gene Logic Inc. BioExpress™ platform and Affymetrix GeneChip® arrays to discover genes differentially expressed in a large series of pancreatic cancers. Biocomputational tools are utilized to determine those genes most highly expressed within pancreatic cancer samples compared to normal pancreatic tissue. Genes found to be significantly expressed in SAGE libraries of normal pancreatic ductal cells are excluded and the expression of selected genes is confirmed by a variety of techniques well known in the art, such as for example, immunohistochemical labeling, in situ hybridization and RT-PCR.

More specifically, the present invention includes the discovery of nucleic acid molecule tumor markers that are differentially present in samples of human cancer patients and control subjects, and the application of this discovery in methods and kits for aiding a human cancer diagnosis. Some of these markers are found at an elevated level and/or more frequently in samples from human cancer patients compared to a control (e.g., early stage of pancreatic cancer which is undetectable by methods in the prior art compared to healthy individuals). Accordingly, the amount of one or more markers found in a test sample compared to a control, or the mere detection of one or more markers in the test sample provides useful information regarding probability of whether a subject being tested has human cancer or not.

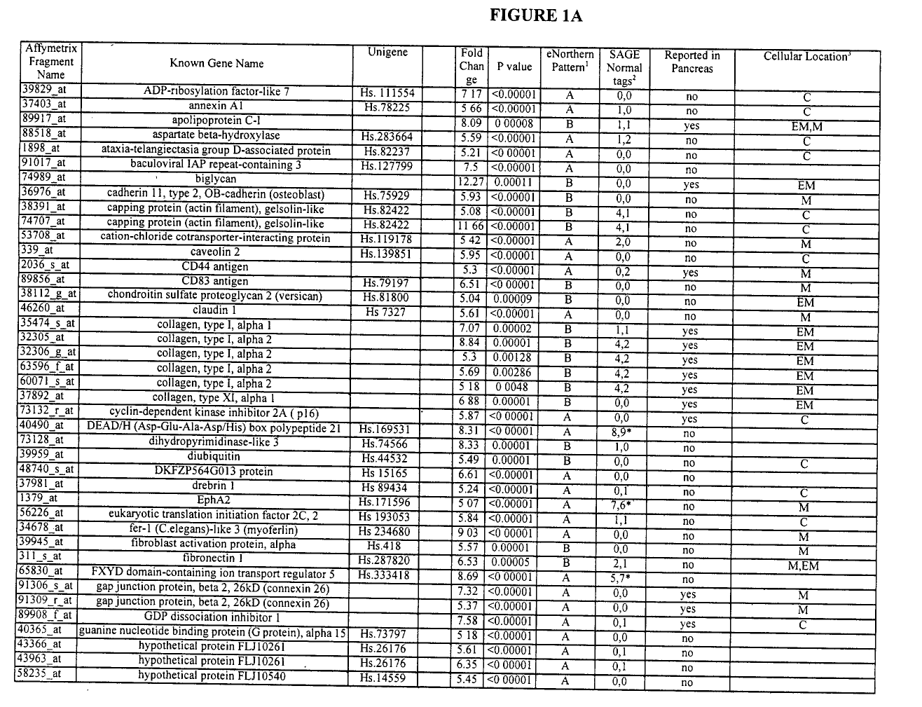

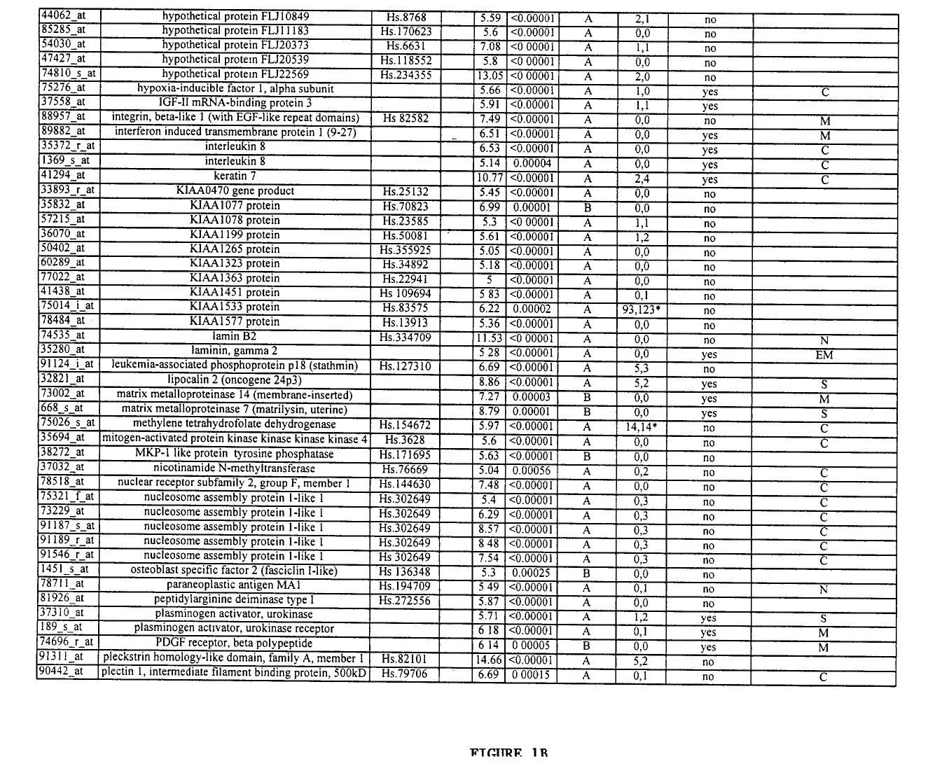

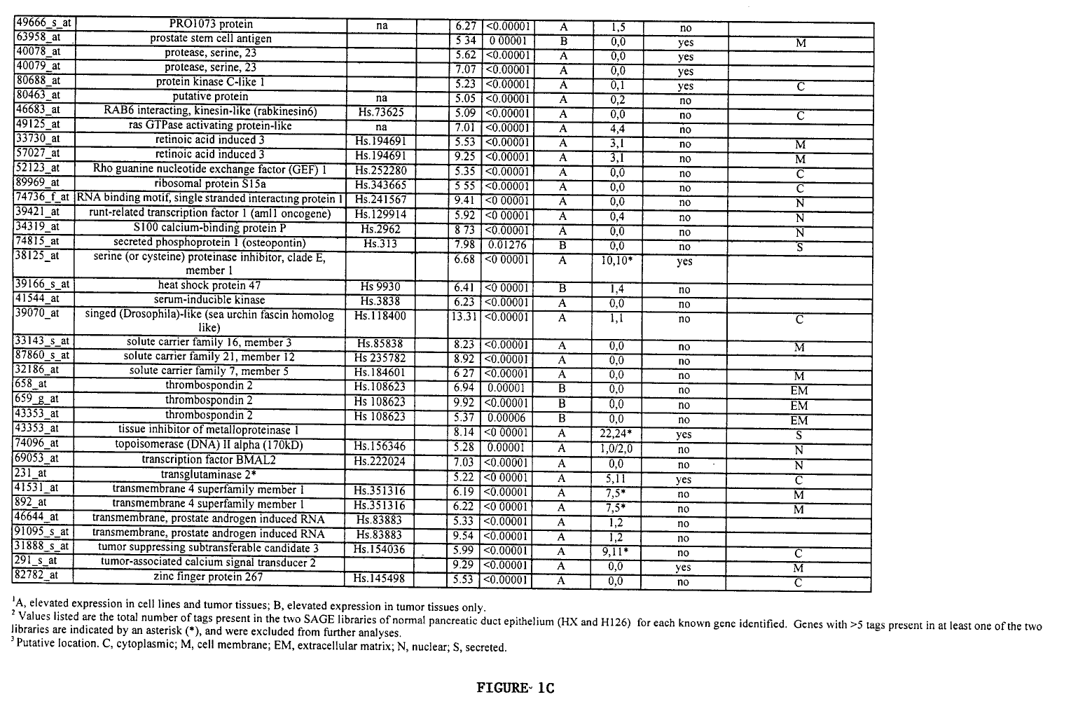

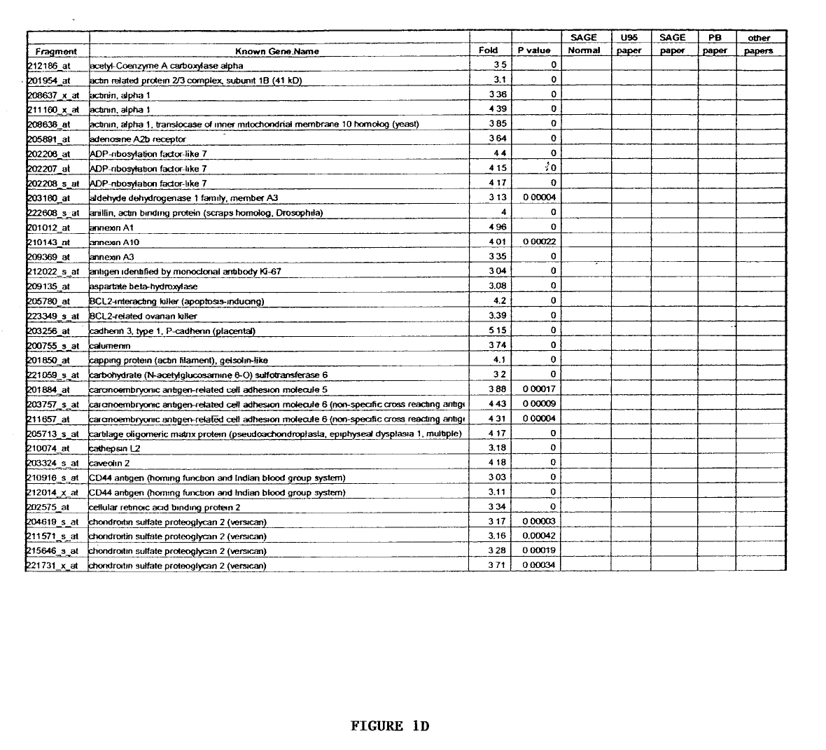

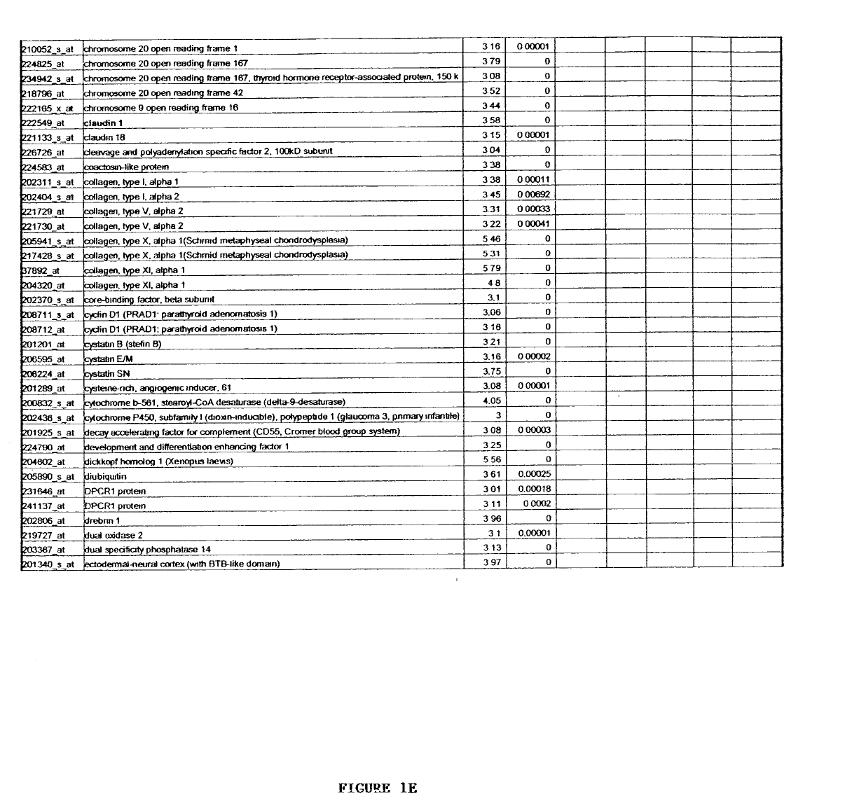

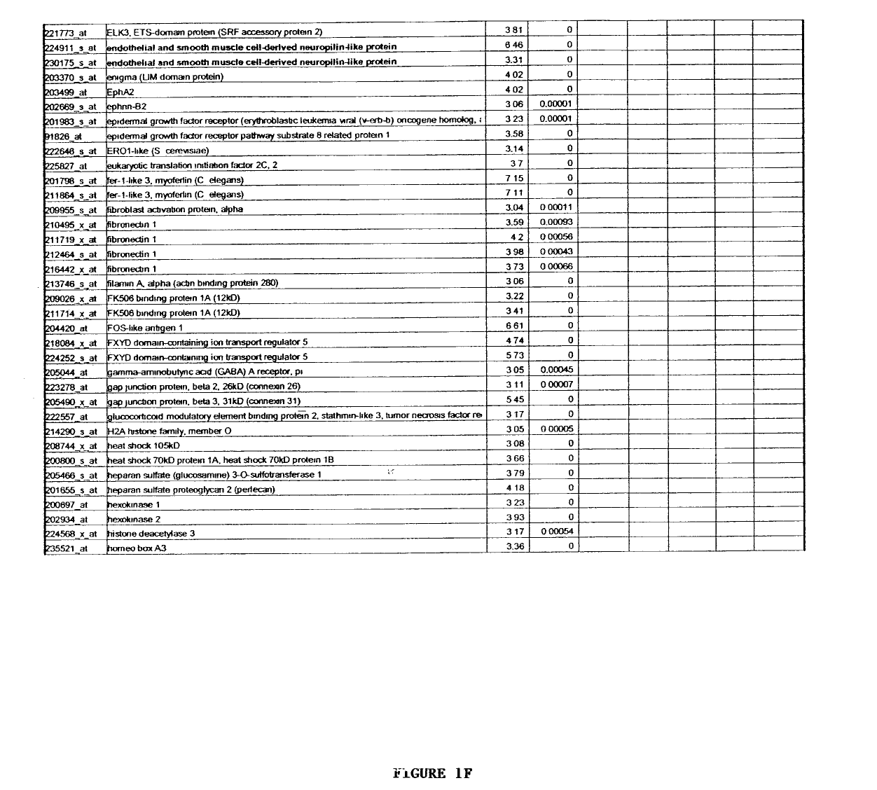

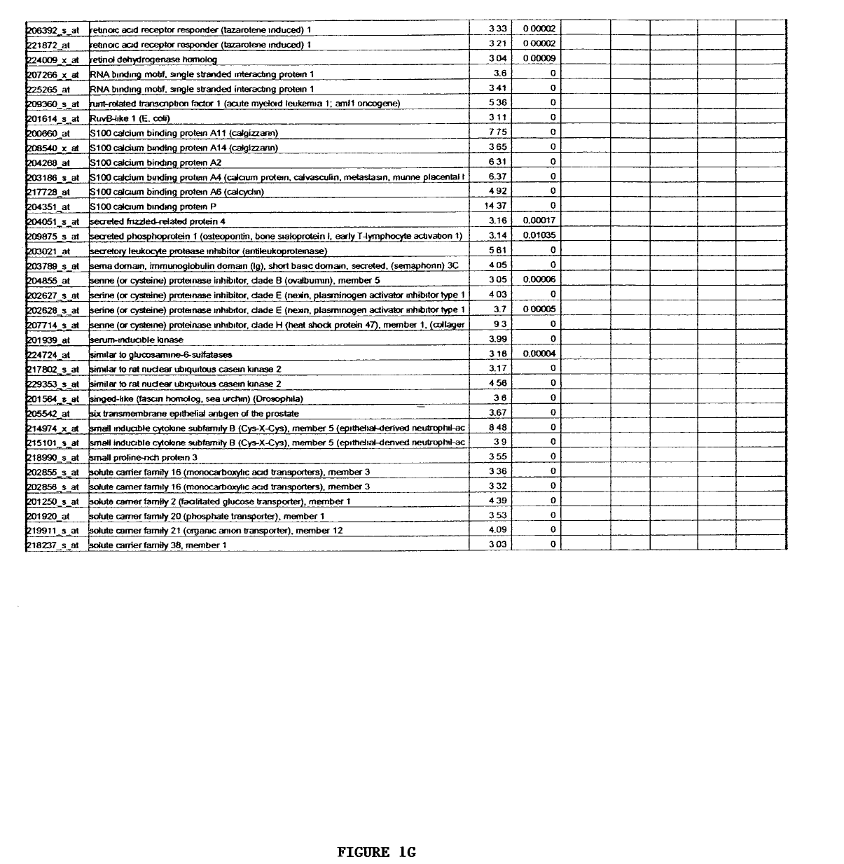

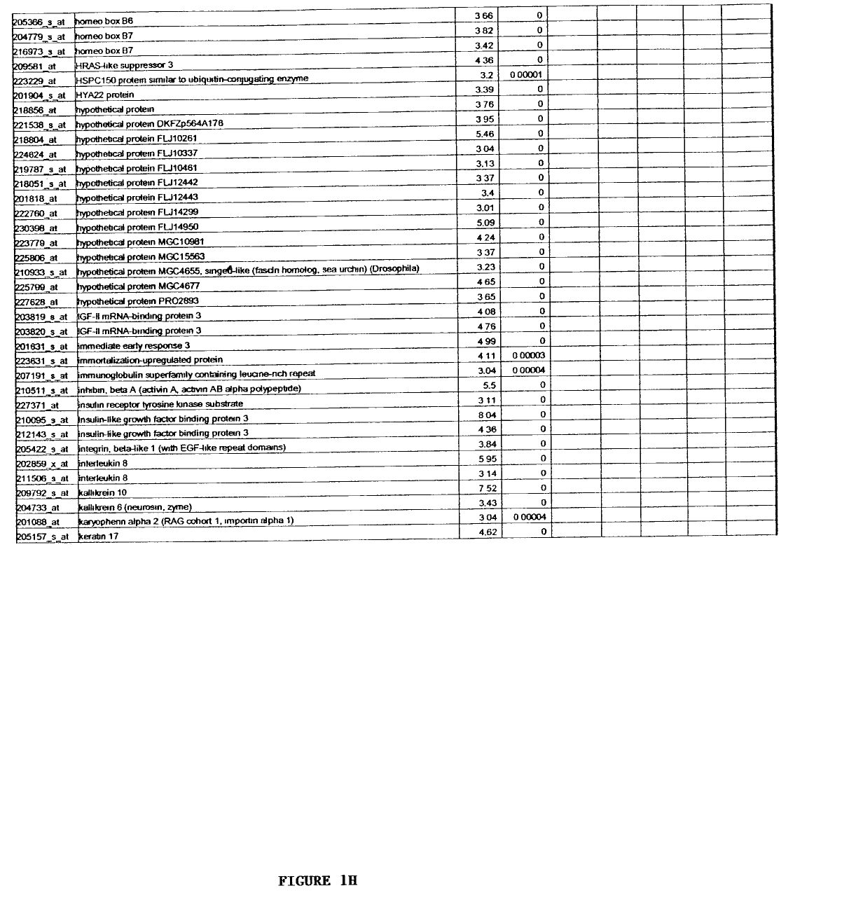

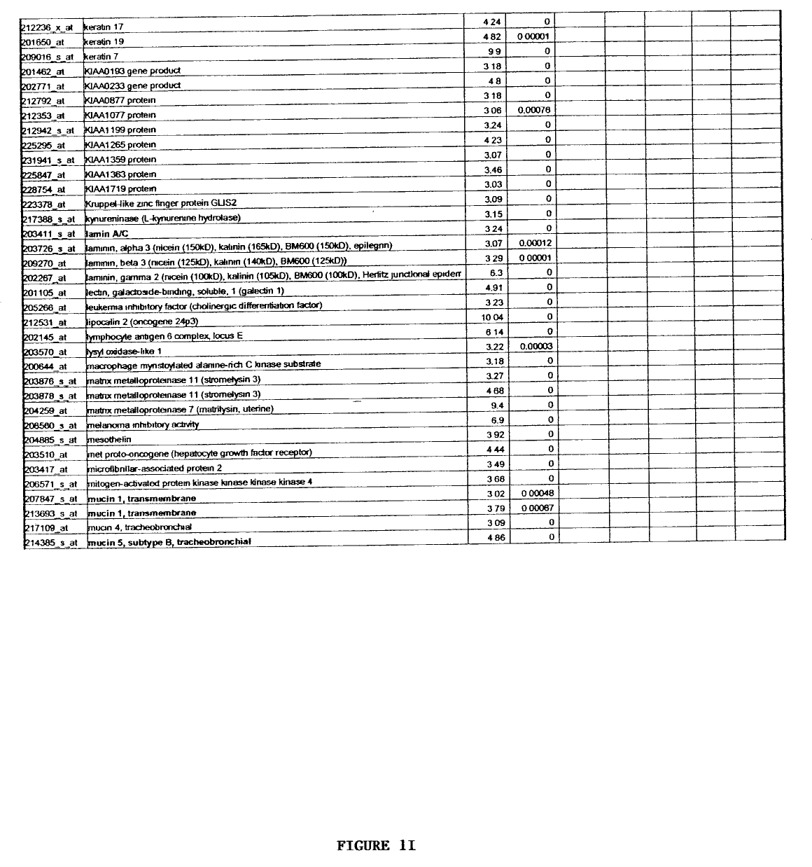

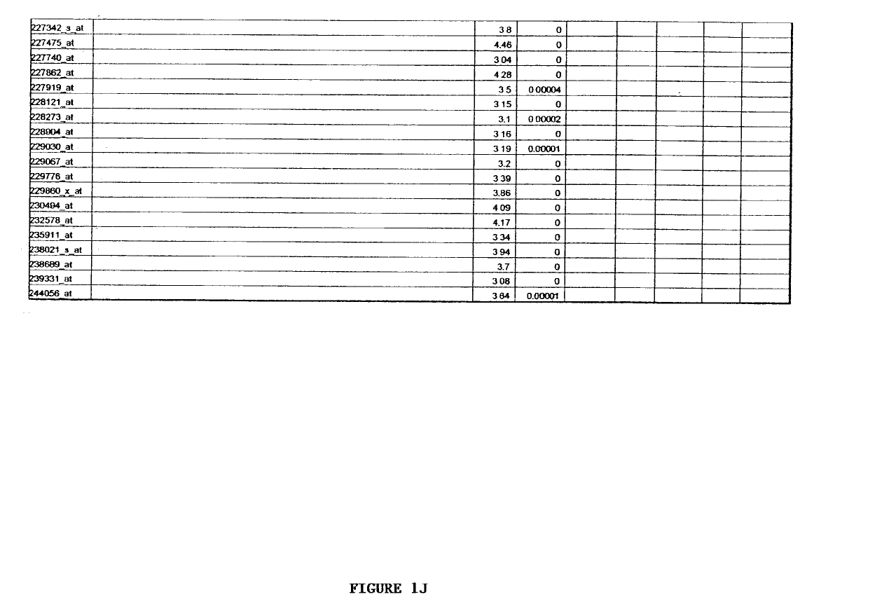

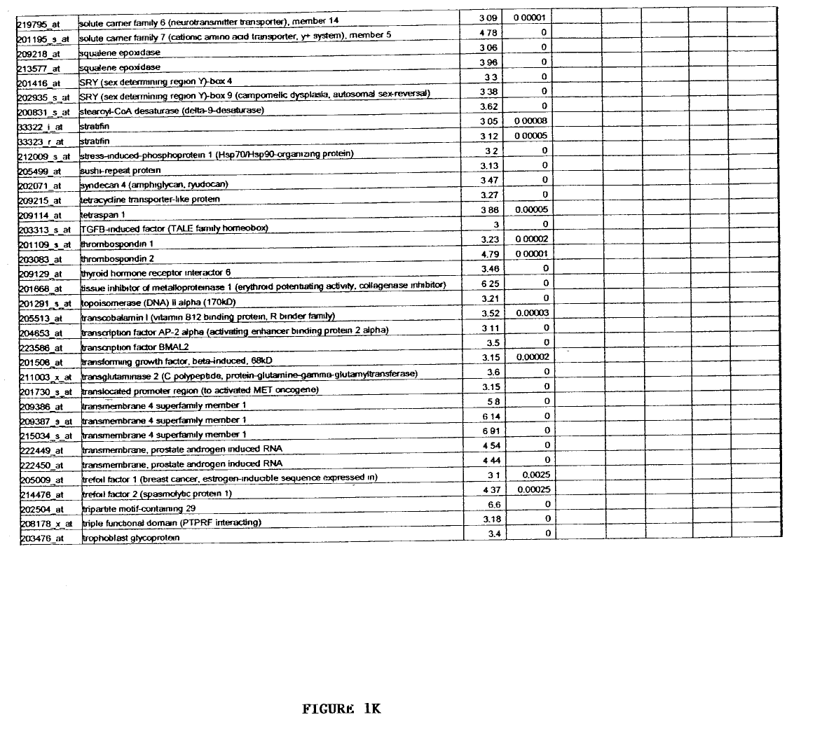

In another preferred embodiment a panel of nucleic acid molecules can be selected from the FIGS. 1A through 1M in any number or combination for the diagnosis of cancer, preferably, pancreatic cancer. The panel can include at least one known tumor marker such as prostate stem cell antigen (PSCA) in combination with about ten or more nucleic acid molecules selected from FIGS. 1A through 1M. A person skilled in the art can easily determine the number of molecules to be used in detecting cancer.

In another aspect of the invention, detection of at least about a five fold increase in the level of expression of nucleic acid molecules as compared to normal controls is diagnostic of a pancreatic cancer. Surprisingly, many of these molecules identified have not been associated with pancreatic cancer and are indicative of the early stages of the disease. The low survival rate of patients diagnosed with pancreatic cancer is due to diagnosis at the late stage of the disease and patients are unresponsive to medical treatment at such a late stage.

As used herein, “pancreatic cancer” is meant to encompass benign or malignant forms of pancreatic cancer, as well as any particular type of cancer arising from cells of the pancreas (e.g., duct cell carcinoma, acinar cell carcinoma, papillary carcinoma, adenosquamous carcinoma, undifferentiated carcinoma, mucinous carcinoma, giant cell carcinoma, mixed type pancreatic cancer, small cell carcinoma, cystadenocarcinoma, unclassified pancreatic cancers, pancreatoblastoma, and papillary-cystic neoplasm, and the like.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, “proliferative growth disorder, “neoplastic disease,” “tumor, “cancer” are used interchangeably as used herein refers to a condition characterized by uncontrolled, abnormal growth of cells. Preferably the cancer to be treated is pancreatic cancer and the abnormal proliferation of cells in the pancreas can be any cell in the organ. Examples of cancer include but are not limited to, carcinoma, blastoma, and sarcoma. As used herein, the term “carcinoma” refers to a new growth that arises from epithelium, found in skin or, more commonly, the lining of body organs.

The term “in need of such treatment” as used herein refers to a judgment made by a care giver such as a physician, nurse, or nurse practitioner in the case of humans that a patient requires or would benefit from treatment. This judgment is made based on a variety of factors that are in the realm of a care giver's expertise, but that include the knowledge that the patient is ill, or will be ill, as the result of a condition that is treatable by the compounds of the invention.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

An “effective amount” of a composition disclosed herein or an agonist thereof, in reference to “inhibiting the cellular proliferation” of a neoplastic cell, is an amount capable of inhibiting, to some extent, the growth of target cells. The term further includes an amount capable of invoking a growth inhibitory, cytostatic and/or cytotoxic effect and/or apoptosis and/or necrosis of the target cells. An “effective amount” of, for example a potential candidate agent that interacts with the nucleic acid molecules described herein, for purposes of inhibiting neoplastic cell growth may be determined empirically and in a routine manner using methods well known in the art.

A “therapeutically effective amount”, in reference to the treatment of neoplastic disease or neoplastic cells, refers to an amount capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

As described in the Examples which follow, sixty nine novel tumor markers have been identified. As an illustrative example, not meant to limit or construe the application in any way, the novel tumor markers were identified as follows. cDNA was prepared from samples of normal pancreas (n=11), normal gastrointestinal mucosa (n=22), resected pancreas cancer tissues (n=14) or pancreas cancer cell lines (n=8), and hybridized to the complete Affymetrix Human Genome U95 GeneChip® set (arrays U95 A,B,C,D and E) for simultaneous analysis of 60,000 fragments, with 12,000 fragments covering full length genes and 48,000 fragments covering ESTs. Genes expressed at least 5 fold greater in the pancreatic cancers compared to normal tissues were identified. SAGE libraries (http://www.ncbl.nlm.nih.gov/SAGE/) of 2 normal pancreatic ductal cell lines (HX and H126) were used to exclude genes expressed in the normal ducts (>5 tags/library). Selected candidate genes were validated by immunohistochemical analysis (n=4), by in situ hybridization (n=1) and RT-PCR (n=8). One-hundred sixty five fragments were identified as 5 fold or greater in expression in pancreas cancer specimens compared to normal, of which 56 were ESTs.

In another example, any markers identified by the present methods can be used in heredity studies. For instance, certain markers may be genetically linked. This can be determined by, e.g., analyzing samples from a population of human cancer patients whose families have a history of human cancer. The results can then be compared with data obtained from, e.g., human cancer patients whose families do not have a history of human cancer. The markers that are genetically linked may be used as a tool to determine if a subject whose family has a history of human cancer is pre-disposed to having human cancer.

In another aspect, the invention provides methods for detecting markers which are differentially present in the samples of a human cancer patient and a control (e.g., an individual in whom human cancer is undetectable). The markers can be detected in a number of biological samples. The sample is preferably a biological fluid, tissue or organ sample. Examples of a biological fluid sample useful in this invention include blood, blood serum, plasma, pancreatic fluids, aspirate, urine, tears, saliva, etc.

The normal pancreas contains a predominance of acinar cells and islets relative to normal duct epithelium. The normal pancreatic duct epithelium is therefore underrepresented in gene expression analyses of bulk normal pancreas. Therefore, in a preferred embodiment, the candidate genes identified by a biochip, such as for example, Affymetrix GeneChip, are further refined to exclude genes highly expressed in cultures of normal pancreatic ductal epithelial cells. For each gene identified as differentially expressed by Affymetrix GeneChip, the corresponding SAGE tag was identified, and the total number of SAGE tags present in the SAGEmap database (http://www.ncbi.nlm.nih.gov/SAGE/) of normal pancreas duct epithelium libraries HX and H126 was determined. Preferably, any gene having at least about five tags in about one of these two SAGE libraries was then excluded from further analysis. Using this approach, for example, 10 genes were identified as having high levels of expression in normal pancreatic duct epithelium (DEAD/H box polypeptide 21, EphA2, FXYD domain-containing ion transport regulator 5, KIAA1577 protein, methylene tetrahydrofolate dehydrogenase, serine/cysteine proteinase inhibitor, clade E1, TIMP1, transglutaminase 2, transmembrane 4 superfamily member 1, and tumor-suppressing subtransferable candidate 3). These genes were excluded, leaving 97 remaining differentially expressed genes (FIGS. 1A through 1M). Thus, based on the results of e-Northern analysis and SAGE filtering, 97 candidate genes were identified as differentially overexpressed in pancreatic cancer. Information regarding the Affymetrix arrays is found in U.S. Pat. Nos. 5,445,934; 5,744,305; 5,700,637; 5,945,334; and EP 619 321; 373, 203, which are hereby incorporated by reference in their entirety.

In a preferred embodiment, nucleic acid sequences with the Unigene accession codes in column 3 of FIGS. 1A to 1C are most preferred. These nucleic acid sequences are identified below in the last column as SEQ ID NOs: 1 through 87. For example,

| Affymetrix | | | SEQ |
|---|---|---|---|
| Fragment | | | ID |
| Name | Known Gene Name | Unigene | NO |
| 39829_at | ADP-ribosylation factor-like 7 | Hs.111554 | 1 |
| 37403_at | annexin A1 | Hs.78225 | 2 |
| 88518_at | aspartate beta-hydroxylase | Hs.283664 | 3 |
| 1898_at | ataxia-telangiectasia group D-associated protein | Hs.82237 | 4 |
| 91017_at | baculoviral IAP repeat-containing 3 | Hs.127799 | 5 |
| 36976_at | cadherin 11, type 2, OB-cadherin (osteoblast) | Hs.75929 | 6 |
| 38391_at | capping protein (actin filament), gelsolin-like | Hs.82422 | 7 |
| 74707_at | capping protein (actin filament), gelsolin-like | Hs.82422 | 8 |
| 53708_at | cation-chloride cotransporter-interacting protein | Hs.119178 | 9 |
| 339_at | caveolin 2 | Hs.139851 | 10 |
| 89856_at | CD83 antigen | Hs.79197 | 11 |
| 38112_g_at | chondroitin sulfate proteoglycan 2 (versican) | Hs 81800 | 12 |
| 46260_at | claudin 1 | Hs.7327 | 13 |
| 40490_at | DEAD/H (Asp-Glu-Ala-Asp/His) box polypeptide 21 | Hs.169531 | 14 |
| 73128_at | dihydropyrimidinase-like 3 | Hs.74566 | 15 |
| 39959_at | diubiquitin | Hs.44532 | 16 |
| 48740_s_at | DKFZP564G013 protein | Hs.15165 | 17 |
| 37981_at | drebrin 1 | Hs.89434 | 18 |
| 1379_at | EphA2 | Hs.171596 | 19 |
| 56226_at | eukaryotic translation initiation factor 2C, 2 | Hs.193053 | 20 |
| 34678_at | fer-1 (C. elegans)-like 3 (myoferlin) | Hs.234680 | 21 |
| 39945_at | fibroblast activation protein, alpha | Hs.418 | 22 |
| 311_s_at | fibronectin 1 | Hs.287820 | 23 |
| 65830_at | FXYD domain-containing ion transport regulator 5 | Hs.333418 | 24 |
| 40365_at | guanine nucleotide binding protein (G protein), alpha 15 | Hs.73797 | 25 |
| 43366_at | hypothetical protein FLJ10261 | Hs.26176 | 26 |
| 43963_at | hypothetical protein FLJ10261 | Hs.26176 | 27 |
| 58235_at | hypothetical protein FLJ10540 | Hs.14559 | 28 |
| 44062_at | hypothetical protein FLJ10849 | Hs.8768 | 29 |
| 85285_at | hypothetical protein FLJ11183 | Hs.170623 | 30 |
| 54030_at | hypothetical protein FLJ20373 | Hs.6631 | 31 |
| 47427_at | hypothetical protein FLJ20539 | Hs.118552 | 32 |
| 74810_s_at | hypothetical protein FLJ22569 | Hs.234355 | 33 |
| 88957_at | integrin, beta-like 1 (with EGF-like repeat domains) | Hs.82582 | 34 |
| 33893_r_at | KIAA0470 gene product | Hs.25132 | 35 |
| 35832_at | KIAA1077 protein | Hs.70823 | 36 |
| 57215_at | KIAA1078 protein | Hs.23585 | 37 |
| 36070_at | KIAA1199 protein | Hs.50081 | 38 |
| 50402_at | KIAA1265 protein | Hs.355925 | 39 |
| 60289_at | KIAA1323 protein | Hs.34892 | 40 |
| 77022_at | KIAA1363 protein | Hs.22941 | 41 |
| 41438_at | KIAA1451 protein | Hs.109694 | 42 |
| 75014_i_at | KIAA1533 protein | Hs.83575 | 43 |
| 78484_at | KIAA1577 protein | Hs.13913 | 44 |
| 74535_at | lamin B2 | Hs.334709 | 45 |
| 91124_i_at | leukemia-associated phosphoprotein p18 (stathmin) | Hs.127310 | 46 |
| 75026_s_at | methylene tetrahydrofolate dehydrogenase | Hs 154672 | 47 |
| 35694_at | mitogen-activated protein kinase kinase kinase kinase 4 | Hs.3628 | 48 |
| 38272_at | MKP-1 like protein tyrosine phosphatase | Hs.171695 | 49 |
| 37032_at | nicotinamide N-methyltransferase | Hs.76669 | 50 |
| 78518_at | nuclear receptor subfamily 2, group F, member 1 | Hs.144630 | 51 |
| 75321_f_at | nucleosome assembly protein 1-like 1 | Hs.302649 | 52 |
| 73229_at | nucleosome assembly protein 1-like 1 | Hs.302649 | 53 |
| 91187_s_at | nucleosome assembly protein 1-like 1 | Hs.302649 | 54 |
| 91189_r_at | nucleosome assembly protein 1-like 1 | Hs.302649 | 55 |
| 91546_r_at | nucleosome assembly protein 1-like 1 | Hs.302649 | 56 |
| 1451_s_at | osteoblast specific factor 2 (fasciclin I-like) | Hs 136348 | 57 |
| 78711_at | paraneoplastic antigen MA1 | Hs.194709 | 58 |
| 81926_at | peptidylarginine deiminase type I | Hs 272556 | 59 |
| 91311_at | pleckstrin homology-like domain, family A, member 1 | Hs.82101 | 60 |
| 90442_at | plectin 1, intermediate filament binding protein, 500 kD | Hs.79706 | 61 |
| 46683_at | RAB6 interacting, kinesin-like (rabkinesin6) | Hs.73625 | 62 |
| 33730_at | retinoic acid induced 3 | Hs.194691 | 63 |
| 57027_at | retinoic acid induced 3 | Hs.194691 | 64 |
| 52123_at | Rho guanine nucleotide exchange factor (GEF) 1 | Hs.252280 | 65 |
| 89969_at | ribosomal protein S15a | Hs.343665 | 66 |
| 74736_f_at | RNA binding motif, single stranded interacting protein 1 | Hs.241567 | 67 |
| 39421_at | runt-related transcription factor 1 (aml1 oncogene) | Hs.129914 | 68 |
| 34319_at | S100 calcium-binding protein P | Hs.2962 | 69 |
| 74815_at | secreted phosphoprotein 1 (osteopontin) | Hs.313 | 70 |
| 39166_s_at | heat shock protein 47 | Hs.9930 | 71 |
| 41544_at | serum-inducible kinase | Hs.3838 | 72 |
| 39070_at | singed (Drosophila)-like (sea urchin fascin homolog like) | Hs 118400 | 73 |
| 33143_s_at | solute carrier family 16, member 3 | Hs.85838 | 74 |
| 87860_s_at | solute carrier family 21, member 12 | Hs.235782 | 75 |
| 32186_at | solute carrier family 7, member 5 | Hs.184601 | 76 |
| 658_at | thrombospondin 2 | Hs.108623 | 77 |
| 659_g_at | thrombospondin 2 | Hs.108623 | 78 |
| 43353_at | thrombospondin 2 | Hs.108623 | 79 |
| 74096_at | topoisomerase (DNA) II alpha (170 kD) | Hs.156346 | 80 |
| 69053_at | transcription factor BMAL2 | Hs.222024 | 81 |
| 41531_at | transmembrane 4 superfamily member 1 | Hs.351316 | 82 |
| 892_at | transmembrane 4 superfamily member 1 | Hs.351316 | 83 |
| 46644_at | transmembrane, prostate androgen induced RNA | Hs.83883 | 84 |
| 91095_s_at | transmembrane, prostate androgen induced RNA | Hs.83883 | 85 |
| 31888_s_at | tumor suppressing subtransferable candidate 3 | Hs.154036 | 86 |
| 82782_at | zinc finger protein 267 | Hs.145498 | 87 |

The Archival UniGene cluster number, for example, for fragment 39829_at (SEQ ID NO 1) is Hs.111554 wherein, the sequence data for the fragment can be retrieved from, for example the NCBI database. The sequence information for SEQ ID NO: 1-87, (or any of the nucleic acid molecules listed in FIGS. 1A to 1M) such as nucleic acid sequence, organism from which the sequences were derived, etc, are available from any public database such as those listed infra.

Serial Analysis of Gene Expression (SAGE), is based on the identification of and characterization of partial, defined sequences of transcripts corresponding to gene segments. These defined transcript sequence “tags” are markers for genes which are expressed in a cell, a tissue, or an extract, for example.

SAGE is based on several principles. First, a short nucleotide sequence tag (9 to 10 bp) contains sufficient information content to uniquely identify a transcript provided it is isolated from a defined position within the transcript. For example, a sequence as short as 9 bp can distinguish 262,144 transcripts (4.sup.9) given a random nucleotide distribution at the tag site, whereas estimates suggest that the human genome encodes about 80,000 to 200,000 transcripts (Fields, et al., Nature Genetics, 7:345 1994). The size of the tag can be shorter for lower eukaryotes or prokaryotes, for example, where the number of transcripts encoded by the genome is lower. For example, a tag as short as 6-7 bp may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. The concept of deriving a defined tag from a sequence in accordance with the present invention is useful in matching tags of samples to a sequence database. In the preferred embodiment, a computer method is used to match a sample sequence with known sequences.

The tags used herein, uniquely identify genes. This is due to their length, and their specific location (3′) in a gene from which they are drawn. The full length genes can be identified by matching the tag to a gene data base member, or by using the tag sequences as probes to physically isolate previously unidentified genes from cDNA libraries. The methods by which genes are isolated from libraries using DNA probes are well known in the art. See, for example, Veculescu et al., Science 270: 484 (1995), and Sambrook et al. (1989), MOLECULAR CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above method but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

Genes whose expression was detected to be increased in pancreatic cancers and which have not been associated with pancreatic cancer are identified in FIGS. 1A through 1M. FIGS. 1A through 1M provides an example of heretofore unknown pancreatic tumor markers. Genes identified included those involved in cell membrane junctions (claudin 1, connexin 26), signal transduction (tumor-associated calcium signal transducer 2, ras GT-Pase-activating protein-like), calcium homeostasis (S100 calcium-binding protein P), cytoskeletal assembly (fascin, keratin 7, rabkinesin6 and pleckstrin), cell surface adhesion and recognition (integrin β-like 1), DNA transcription (topoisomerase IIα, transcription factor BMAL2, and AML1), DNA repair (ATDC), or extracellular matrix remodeling and function (collagens 1α1, 1α2, and X1α1, heat shock protein 47, MMP14, and MMP7). The cellular localization of the corresponding gene products was also determined using the online database OMIM available through the NCBI web site (http://www.ncbi.nlm.nih.gov/entrez/query). Genes were found to encode membrane-bound proteins (prostate stem cell antigen, OB-cadherin), cytoplasmic proteins (fascin, ATDC), nuclear proteins (topoisomerase IIα, paraneoplastic antigen MA1), as well as extracellular proteins, such as those involved in extracellular matrix homeostasis (hsp47, thrombospondin 2) or secreted protein products (osteopontin).

In a preferred embodiment, Expressed Sequenced Tags (ESTs), can also be used to identify nucleic acid molecules which are over expressed in a cancer cell. ESTs from a variety of databases can be indentified. For example, preferred databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet, at, for example, ncbi.nlm.nih.gov/Omim/. CGAP, which is an interdisciplinary program to establish the information and technological tools required to decipher the molecular anatomy of a cancer cell. CGAP can be accessed through the world wide web of the Internet, at, for example, ncbi.nlm.nih.gov/ncicgap/. Some of these databases may contain complete or partial nucleotide sequences. In addition, alternative transcript forms can also be selected from private genetic databases. Alternatively, nucleic acid molecules can be selected from available publications or can be determined especially for use in connection with the present invention.

Alternative transcript forms can be generated from individual ESTs which are within each of the databases by computer software which generates contiguous sequences. In another embodiment of the present invention, the nucleotide sequence of the nucleic acid molecule is determined by assembling a plurality of overlapping ESTs. The EST database (dbEST), which is known and available to those skilled in the art, comprises approximately one million different human mRNA sequences comprising from about 500 to 1000 nucleotides, and various numbers of ESTs from a number of different organisms. dbEST can be accessed through the world wide web of the Internet, at, for example, ncbi.nlm.nih.gov/dbEST/index.html. These sequences are derived from a cloning strategy that uses cDNA expression clones for genome sequencing. ESTs have applications in the discovery of new genes, mapping of genomes, and identification of coding regions in genomic sequences. Another important feature of EST sequence information that is becoming rapidly available is tissue-specific gene expression data. This can be extremely useful in targeting selective gene(s) for therapeutic intervention. Since EST sequences are relatively short, they must be assembled in order to provide a complete sequence. Because every available clone is sequenced, it results in a number of overlapping regions being reported in the database. The end result is the elicitation of alternative transcript forms from, for example, normal cells and cancer cells.

Assembly of overlapping ESTs extended along both the 5′ and 3′ directions results in a full-length “virtual transcript.” The resultant virtual transcript may represent an already characterized nucleic acid or may be a novel nucleic acid with no known biological function. The Institute for Genomic Research (TIGR) Human Genome Index (HGI) database, which is known and available to those skilled in the art, contains a list of human transcripts. TIGR can be accessed through the world wide web of the Internet, at, for example, tigr.org. Transcripts can be generated in this manner using TIGR-Assembler, an engine to build virtual transcripts and which is known and available to those skilled in the art. TIGR-Assembler is a tool for assembling large sets of overlapping sequence data such as ESTs, BACs, or small genomes, and can be used to assemble eukaryotic or prokaryotic sequences. TIGR-Assembler is described in, for example, Sutton, et al., Genome Science & Tech., 1995, 1, 9-19, which is incorporated herein by reference in its entirety, and can be accessed through the file transfer program of the Internet, at, for example, tigr.org/pub/software/TIGR. assembler. In addition, GLAXO-MRC, which is known and available to those skilled in the art, is another protocol for constructing virtual transcripts. In addition, “Find Neighbors and Assemble EST Blast” protocol, which runs on a UNIX platform, has been developed by Applicants to construct virtual transcripts. PHRAP is used for sequence assembly within Find Neighbors and Assemble EST Blast. PHRAP can be accessed through the world wide web of the Internet, at, for example, chimera.biotech.washington.edu/uwgc/tools/phrap.htm. Identification of ESTs and generation of contiguous ESTs to form full length RNA molecules is described in detail in U.S. application Ser. No. 09/076,440, which is incorporated herein by reference in its entirety.

In yet another aspect, variants of the nucleic acid molecules as identified in FIGS. 1A through 1M can be used to detect pancreatic cancers. An “allele” or “variant” is an alternative form of a gene. Of particular utility in the invention are variants of the genes encoding any potential pancreatic tumor markers identified by the methods of this invention. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

To further identify variant nucleic acid-molecules which can detect, for example, pancreatic cancer at an early stage, nucleic acid molecules can be grouped into sets depending on the homology, for example. The members of a set of nucleic acid molecules are compared. Preferably, the set of nucleic acid molecules is a set of alternative transcript forms of nucleic acid. Preferably, the members of the set of alternative transcript forms of nucleic acids include at least one member which is associated, or whose encoded protein is associated, with a disease state or biological condition. For example, a set of nucleic acid molecules for the KIAA genes are compared (see FIGS. 1A through 1M). At least one of the members of the set of nucleic acid molecule alternative transcript forms is associated with cancer, as described above. Thus, comparison of the members of the set of nucleic acid molecules results in the identification of at least one alternative transcript form of nucleic acid molecule which is associated, or whose encoded protein is associated, with a disease state or biological condition. In a preferred embodiment of the invention, the members of the set of nucleic acid molecules are from a common gene. In another embodiment of the invention, the members of the set of nucleic acid molecules are from a plurality of genes. In another embodiment of the invention, the members of the set of nucleic acid molecules are from different taxonomic species. Nucleotide sequences of a plurality of nucleic acids from different taxonomic species can be identified by performing a sequence similarity search, an ortholog search, or both, such searches being known to persons of ordinary skill in the art.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

Preferably, the plurality of nucleic acids from different taxonomic species which have homology to the target nucleic acid, as described above in the sequence similarity search, are further delineated so as to find orthologs of the target nucleic acid therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing an ortholog search, an exhaustive list of homologous sequences from as diverse organisms as possible is obtained. Subsequently, these sequences are analyzed to select the best representative sequence that fits the criteria for being an ortholog. An ortholog search can be performed by programs available to those skilled in the art including, for example, Compare. Preferably, an ortholog search is performed with access to complete and parsed GenBank annotations for each of the sequences. Currently, the records obtained from GenBank are “flat-files”, and are not ideally suited for automated analysis. Preferably, the ortholog search is performed using a Q-Compare program. Preferred steps of the Q-Compare protocol are described in the flowchart set forth in U.S. Pat. No. 6,221,587, incorporated herein by reference.

Preferably, interspecies sequence comparison is performed using Compare, which is available and known to those skilled in the art. Compare is a GCG tool that allows pair-wise comparisons of sequences using a window/stringency criterion. Compare produces an output file containing points where matches of specified quality are found. These can be plotted with another GCG tool, DotPlot.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) or MacPherson et al. (1991) and (1994), supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (procaryotic or eucaryotic) for replication and amplification. The DNA so amplified can be isolated from the cell by methods well known to those of skill in the art. A process for obtaining polynucleotides by this method is further provided herein as well as the polynucleotides so obtained.

The terms “nucleic acid molecule” and “tumor marker” or “polynucleotide” will be used interchangeably throughout the specification, unless otherwise specified. As used herein, “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

In an embodiment of the invention the presence of the one or more nucleic acid molecules is correlated to a sample of a normal subject. The sample is preferably obtained from a mammal suspected of having a proliferative cell growth disorder, in particular, a pancreatic cancer. Preferably, a nucleic acid molecule that is indicative of a pancreatic cancer comprises a sequence having at least about 80% sequence identity to a molecule identified in any of FIGS. 1A through 1M, more preferably the nucleic acid molecule comprises a sequence having at least about 90% sequence identity to a molecule identified in any of FIGS. 1A through 1M, most preferably the nucleic acid molecule comprises a sequence having at least about 95% sequence identity to a molecule identified in any of FIGS. 1A through 1M.

In another preferred embodiment, the nucleic acid molecule is expressed at a higher level in a patient with cancer as compared to expression levels in a normal individual. Preferably the nucleic acid molecule is expressed at least about 10 fold higher in a patient with cancer as compared to expression in a normal individual, more preferably the nucleic acid molecule is expressed at least about 10 fold higher in a patient with cancer as compared to expression in a normal individual, most preferably the nucleic acid molecule is expressed at least about 5 fold higher in a patient with cancer as compared to expression in a normal individual.

Percent identity and similarity between two sequences (nucleic acid or polypeptide) can be determined using a mathematical algorithm (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

To determine the percent identity of two amino acid sequences or of two nucleic acid sequences, the sequences are aligned for optimal comparison purposes (e.g., gaps are introduced in one or both of a first and a second amino acid or nucleic acid sequence for optimal alignment and non-homologous sequences can be disregarded for comparison purposes). The percent identity between the two sequences is a function of the number of identical positions shared by the sequences, taking into account the number of gaps, and the length of each gap which need to be introduced for optimal alignment of the two sequences. The amino acid residues or nucleotides at corresponding amino acid positions or nucleotide positions, respectively, are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

A “comparison window” refers to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

For example, the percent identity between two amino acid sequences can be determined using the Needleman and Wunsch algorithm (J. Mol. Biol. (48): 444-453, 1970) which is part of the GAP program in the GCG software package (available at http://www.gcg.com), by the local homology algorithm of Smith & Waterman (Adv. Appl. Math. 2: 482, 1981), by the search for similarity methods of Pearson & Lipman (Proc. Natl. Acad. Sci. USA 85: 2444, 1988) and Altschul, et al. (Nucleic Acids Res. 25(17): 3389-3402, 1997), by computerized implementations of these algorithms (GAP, BESTFIT, FASTA, and BLAST in the Wisconsin Genetics Software Package (available from, Genetics Computer Group, 575 Science Dr., Madison, Wis.), or by manual alignment and visual inspection (see, e.g., Ausubel et al., supra). Gap parameters can be modified to suit a user's needs. For example, when employing the GCG software package, a NWSgapdna.CMP matrix and a gap weight of 40, 50, 60, 70, or 80 and a length weight of 1, 2, 3, 4, 5, or 6 can be used. Examplary gap weights using a Blossom 62 matrix or a PAM250 matrix, are 16, 14, 12, 10, 8, 6, or 4, while exemplary length weights are 1, 2, 3, 4, 5, or 6. The GCG software package can be used to determine percent identity between nucleic acid sequences. The percent identity between two amino acid or nucleotide sequences also can be determined using the algorithm of E. Myers and W. Miller (CABIOS 4: 11-17, 1989) which has been incorporated into the ALIGN program (version 2.0), using a PAM120 weight residue table, a gap length penalty of 12 and a gap penalty of 4.

The nucleic acid and protein sequences of the present invention can further be used as query sequences to perform a search against sequence databases to, for example, identify other family members or related sequences. Such searches can be performed using the NBLAST and XBLAST programs (version 2.0) of Altschul, et al. (J. Mol. Biol. 215: 403-10, 1990). BLAST nucleotide searches can be performed with the NBLAST program, with exemplary scores=100, and wordlengths=12 to obtain nucleotide sequences homologous to or with sufficient percent identity to the nucleic acid molecules of the invention. BLAST protein searches can be performed with the XBLAST program, with exemplary scores=50 and wordlengths=3 to obtain amino acid sequences sufficiently homologous to or with sufficient % identity to the proteins of the invention. To obtain gapped alignments for comparison purposes, gapped BLAST can be used as described in Altschul et al. (Nucleic Acids Res. 25(17): 3389-3402, 1997). When using BLAST and gapped BLAST programs, the default parameters of the respective programs (e.g., XBLAST and NBLAST) can be used.

The invention also encompasses polypeptides corresponding to a nucleic acid molecule product such as those identified in FIGS. 1A through 1M, which comprises conservative substitutions that are phenotypically silent. Such substitutions are those that substitute a given amino acid in a polypeptide by another amino acid of like characteristics. Guidance concerning amino acid changes which are likely to be phenotypically silent may be found in Bowie et al., Science 247: 1306-1310,1990, for example. Conservative substitution tables providing functionally similar amino acids are well known in the art (see, e.g., Henikoff and Henikoff, Proc. Natl. Acad. Sci. USA 89: 10915-10919,1992) and in the table below.

Conservative Amino Acid Substitutions

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

A significant “fragment” in a nucleic acid context is a contiguous segment of at least about 17 nucleotides, generally at least 20 nucleotides, more generally at least 23 nucleotides, ordinarily at least 26 nucleotides, more ordinarily at least 29 nucleotides, often at least 32 nucleotides, more often at least 35 nucleotides, typically at least 38 nucleotides, more typically at least 41 nucleotides, usually at least 44 nucleotides, more usually at least 47 nucleotides, preferably at least 50 nucleotides, more preferably at least 53 nucleotides, and in particularly preferred embodiments will be at least 56 or more nucleotides. Additional preferred embodiments will include lengths in excess of those numbers, e.g., 63, 72, 87, 96, 105, 117, etc. Said fragments may have termini at any pairs of locations, but especially at boundaries between structural domains, e.g., membrane spanning portions.

Homologous nucleic acid sequences, when compared, exhibit significant sequence identity or similarity. The standards for homology in nucleic acids are either measures for homology generally used in the art by sequence comparison or based upon hybridization conditions. The hybridization conditions are described in greater detail below.

As used herein, “substantial homology” in the nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial homology exists when the segments will hybridize under selective hybridization conditions, to a strand, or its complement, typically using a fragment derived from FIGS. 1A through 1M, e.g., 39829_at. Typically, selective hybridization will occur when there is at least about 55% homology over a stretch of at least about 14 nucleotides, preferably at least about 65%, more preferably at least about 75%, and most preferably at least about 90%. See, Kanehisa (1984) Nuc. Acids Res. 12:203-213. The length of homology comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. The endpoints of the segments may be at many different pair combinations.

Stringent conditions, in referring to homology in the hybridization context, will be stringent combined conditions of salt, temperature, organic solvents, and other parameters, typically those controlled in hybridization reactions. Stringent temperature conditions will usually include temperatures in excess of about 30° C., more usually in excess of about 37° C., typically in excess of about 45° C., more typically in excess of about 55° C., preferably in excess of about 65° C., and more preferably in excess of about 70° C. Stringent salt conditions will ordinarily be less than about 1000 mM, usually less than about 500 mM, more usually less than about 400 mM, typically less than about 300 mM, preferably less than about 200 mM, and more preferably less than about 150 mM. However, the combination of parameters is much more important than the measure of any single parameter. See, e.g., Wetmur and Davidson (1968) J. Mol. Biol. 31:349-370.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

The terms “specific binding” or “specifically binding”, as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

In another preferred embodiment, the identified genes and gene products can be used to generate antibodies and/or lymphocytes that can function to block or lyse a cell with the overexpressed molecules. Methods for generation of antibodies are well known in the art. Antibodies that specifically bind to a tumor marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Antibodies can be used in immunoassays to detect, for example, cells expressing the gene products of genes listed in FIGS. 1A through 1M. This is useful in further aiding in the diagnosis of pancreatic cancer at an early stage. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Immunoassay” is an assay that uses an antibody to specifically bind an antigen (e.g., a marker). The immunoassay is characterized by the use of specific binding properties of a particular antibody to isolate, target, and/or quantify the antigen.

After the antibody is provided, a tumor marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological sample taken from a subject.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Immunoassays can be used to determine presence or absence of a marker in a sample as well as the quantity of a marker in a sample. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro.

In accordance with the invention, lymphocytes such as T lymphocytes from a patient may be cultured ex-vivo with a tumor marker that is expressed on the cell surface and re-infused into a patient. Such methods are well known in the art, see for example U.S. Pat. No. 6,225,042, which is hereby incorporated in its entirety by reference. Preferably the T lymphocytes are CD8 positive lymphocytes. In another variation, the invention relates to treating conditions in patients and specifically killing target cells in a human patient (see for example U.S. Pat. No. 6,225,042). The method comprises (1) obtaining a fluid sample containing resting or naive CD8 cells from the patient; (2) contacting, in vitro, the CD8 cells with an antigen-presenting matrix for a time period sufficient to activate, in an antigen-specific manner, the CD8 cells; and (3) administering the activated CD8 cells to the patient.

In accordance with the invention; therapeutic treatment combines the cytotoxic capability of T cells and the specificity of antibodies to augment the cytotoxic capacity of T cells to lyse tumors.

In a preferred embodiment, the invention provides a vector for administering to a patient suffering from or susceptible to cancer, especially pancreatic cancer. In accordance with the invention a vector comprising one or more nucleic acid molecules, or variants thereof, where the nucleic acid molecule comprises a sequence corresponding to a molecule identified in any of FIGS. 1A through 1M. The vector preferably comprises nucleic acid molecules comprising a sequence having at least about 80% sequence identity to a molecule identified in any of FIGS. 1A through 1M, more preferable the vector preferably comprises nucleic acid molecules comprising a sequence having at least about 90% sequence identity to a molecule identified in any of FIGS. 1A through 1M, most preferable more preferable the vector preferably comprises nucleic acid molecules comprising a sequence having at least about 95% sequence identity to a molecule identified in any of FIGS. 1A through 1M. Preferably, the vector generates an in vivo immune response resulting in the cytolysis of a cell overexpressing the product of the nucleic acid molecules.

By “patient” herein is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Activity”, “activation” or “augmentation” is the ability of immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

Immune cell activity that may be measured include, but is not limited to, (1) cell proliferation by measuring the cell or DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

As discussed above, a preferred use of nucleic acid sequences identified in the present invention, is for the generation of treatments that lyse for example, pancreatic cancer cells. The nucleic acid molecules can be expressed by a vector containing a DNA segment encoding the wild-type, alleles, variants, mutations or fragments of the genes. Mutations and alleles of the nucleic acid molecules are also preferably used in the construction of a vector for use in treatment. The vector comprising the desired nucleic acid sequence for conferring resistance to, for example, pancreatic cancer, preferably has at least one such nucleic acid sequence. Alternatively, the vector may be comprised of more than one such nucleic acid sequence, or combinations of allelic variants. The vector can also be comprised of cassettes of different allelic variants or wild type nucleic acid molecules.

According to the present invention, the coding sequence on the plasmid that encodes the nucleic acid molecules is provided with a coding sequence that encodes an amino acid sequence whose presence on the protein results in a specific intracellular localization of the expressed protein. The nucleotide sequences that encode amino acid sequences which direct intracellular protein trafficking and which are included in the coding sequences of immunogenic proteins that are included in plasmid constructs used as DNA therapeutic compositions direct localization to specific areas in the cells which result in enhancement or activation of the immune response.

Introducing the genes, fragments or alleles thereof, into an individual can include use of vectors, liposomes, naked DNA, adjuvant-assisted DNA, gene gun, catheters, etc. Vectors include chemical conjugates such as described in WO 93/04701, which has a targeting moiety (e.g. a ligand to a cellular surface receptor), and a nucleic acid binding moiety (e.g. polylysine), viral vector (e.g. a DNA or RNA viral vector), fusion proteins such as described in PCT/US95/02140 (WO 95/22618) which is a fusion protein containing a target moiety (e.g. an antibody specific for a target cell) and a nucleic acid binding moiety (e.g. a protamine), plasmids, phage etc. The vectors can be chromosomal, non-chromosomal or synthetic.

It is a preferred embodiment of this invention that the choice of cells for delivery of the nucleic acid molecules include embryonic stem cells, hematopoietic cells which can be differentiated into monocytes/macrophages using cytokines known in the art and delivery of these cells into an individual.

Preferred nucleic acid sequences that encode for a modified nucleic acid sequences may suitably comprise any of the molecules identified in FIGS. 1A through 1M, as well as sequences that have a substantial sequence identity to FIGS. 1A through 1M, e.g. at least about 70, 75, 80, 85, 90 or 95 percent sequence identity to any one or more of those sequences.

Preferred vectors include viral vectors, fusion proteins and chemical conjugates. Retroviral vectors include moloney murine leukemia viruses. DNA viral vectors are preferred. Viral vectors can be chosen to introduce the genes to cells of choice. Such vectors include pox vectors such as orthopox or avipox vectors, herpesvirus vectors such as herpes simplex I virus (HSV) vector (Geller et al., 1995, J. Neurochem. 64: 487; Lim et al., 1995, in DNA Cloning: Mammalian Systems, D. Glover, ed., Oxford Univ. Press, Oxford, England; Geller et al., 1990, Proc. Natl. Acad. Sci. U.S.A. 87: 1149), adenovirus vectors (LeGal LaSalle et al., 1993, Science 259: 988; Davidson et al., 1993, Nat. Genet. 3: 219; Yang et al., 1995, J. Virol. 69: 2004) and adeno-associated virus vectors (Kaplitt et al., 1994, Nat. Genet. 8: 148).

Pox viral vectors introduce the gene into the cells cytoplasm. Avipox virus vectors result in only short term expression of the nucleic acid. Adenovirus vectors, adeno-associated virus vectors and herpes simplex virus vectors are preferred for introducing the nucleic acid into neural cells. The adenovirus vector results in a shorter term expression (about 2 months) than adeno-associated virus (about 4 months), which in turn is shorter than HSV vectors. The vectors can be introduced by standard techniques, e.g. infection, transfection, transduction or transformation. Examples of modes of gene transfer include for example, naked DNA calcium phosphate precipitation, DEAE dextran, electroporation, protoplast fusion, lipofection, cell microinjection and viral vectors.

The vector can be employed to target essentially any desired target cell. For example, stereotaxic injection can be used to direct the vectors (e.g. adenovirus, HSV) to a desired location. Other methods that can be used include catheters, intravenous, parenteral, intraperitoneal, and subcutaneous injection, and oral or other known routes of administration.

Another preferred method is DNA immunization. DNA immunization employs the subcutaneous injection of a plasmid DNA (pDNA) vector encoding a tumor marker. The pDNA sequence is taken up by antigen presenting cells (APC). Once inside the cell, the DNA encoding protein is transcribed and translated and presented to lymphocytes.

Genetic constructs comprise a nucleotide sequence that encodes the nucleic acid sequence of choice and preferably includes an intracellular trafficking sequence operably linked to regulatory elements needed for gene expression.

When taken up by a cell, the genetic construct(s) may remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal.

Genetic constructs include regulatory elements necessary for gene expression of a nucleic acid molecule. The elements include: a promoter, an initiation codon, a stop codon, and a polyadenylation signal. In addition, enhancers may be required for gene expression of the sequence of choice, for example, the nucleic acid sequences identified in FIGS. 1A through 1M, gene, alleles or fragments thereof. It is necessary that these elements be operably linked to the sequence that encodes the desired proteins and that the regulatory elements are operable in the individual to whom they are administered.

Initiation codons and stop codons are generally considered to be part of a nucleotide sequence that encodes the immunogenic target protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. For example, plasmids pCEP4 and pREP4 from Invitrogen (San Diego, Calif.) contain the Epstein Barr virus origin of replication and nuclear antigen EBNA-1 coding region which produces-high copy episomal replication without integration.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs which are functional in the cells.

The method of the present invention comprises the steps of administering nucleic acid molecules to tissue of the individual. In some preferred embodiments, the nucleic acid molecules are administered intramuscularly, intranasally, intraperatoneally, subcutaneously, intradermally, or topically or by lavage to mucosal tissue selected from the group consisting of vaginal, rectal, urethral, buccal and sublingual.

In some embodiments, the nucleic acid molecule is delivered to the cells in conjunction with administration of a facilitating agent. Facilitating agents are also referred to as polynucleotide function enhancers or genetic vaccine facilitator agents. Facilitating agents are described in e.g. International Application No. PCT/US94/00899 filed Jan. 26, 1994 and International Application No. PCT/US95/04071 filed Mar. 30, 1995, both incorporated herein by reference. Facilitating agents which are administered in conjunction with nucleic acid molecules may be administered as a mixture with the nucleic acid molecule or administered separately simultaneously, before or after administration of nucleic acid molecules.

In some preferred embodiments, the genetic constructs of the invention are formulated with or administered in conjunction with a facilitator selected from the group consisting of, for example, benzoic acid esters, anilides, amidines, urethans and the hydrochloride salts thereof such as those of the family of local anesthetics. The facilitating agent is administered prior to, simultaneously with or subsequent to the genetic construct. The facilitating agent and the genetic construct may be formulated in the same composition.

In some embodiments of the invention, the individual is first subject to injection of the facilitator prior to administration of the genetic construct. That is, for example, up to a about a week to ten days prior to administration of the genetic construct, the individual is first injected with the facilitator. In some embodiments, the individual is injected with the facilitator about 1 to 5 days; in some embodiments 24 hours, before or after administration of the genetic construct. Alternatively, if used at all, the facilitator is administered simultaneously, minutes before or after administration of the genetic construct. Accordingly, the facilitator and, the genetic construct may be combined to form a single pharmaceutical composition.

In some embodiments, the genetic constructs are administered free of facilitating agents, that is in formulations free from facilitating agents using administration protocols in which the genetic constructions are not administered in conjunction with the administration of facilitating agents.

Nucleic acid molecules which are delivered to cells according to the invention may serve as genetic templates for proteins that function as prophylactic and/or therapeutic immunizing agents. In preferred embodiments, the nucleic acid molecules comprise the necessary regulatory sequences for transcription and translation of the coding region in the cells of the animal.

In yet another aspect, the invention provides kits for aiding a diagnosis of human cancer, wherein the kits can be used to detect the markers of the present invention. For example, the kits can be used to detect any one or more of the markers described herein, which markers are differentially present in samples of a human cancer patient and normal subjects. The kits of the invention have many applications. For example, the kits can be used to differentiate if a subject has human pancreatic cancer or has a negative diagnosis and the stage of the cancer, preferably the earliest stage, thus aiding a human cancer diagnosis. In another example, the kits can be used to identify compounds that modulate expression of one or more of the markers in in vitro or in vivo animal models for human cancer.

In one embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry.

Optionally, the kit can further comprise instructions for suitable operational parameters in the form of a label or a separate insert. For example, the kit may have standard instructions informing a consumer how to wash the probe after a sample of blood serum is contacted on the probe. In another example, the kit may have instructions for pre-fractionating a sample to reduce complexity of molecules in the sample. In another example, the kit may have instructions for automating the fractionation or other processes.

In another embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.). Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with an early diagnosis of human pancreatic cancer.

All documents mentioned herein are incorporated herein by reference in their entirety.

The following non-limiting examples are illustrative of the invention.

### EXAMPLES

In the following examples, the following Materials and Methods were used.

Materials and Methods

Cell Lines.

Human pancreatic cancer cell lines AsPC1, BxPC3, CAPAN1, CAPAN2, CfPACI , Colo357, Hs766T, MiaPaca2, Panc-1 and Su86.86, and human pancreatic normal duct epithelial line H6C7, were obtained from the American Type Culture Collection, Rockville, Md. PL cell lines (PL1-6, PL8-14) were low-passage pancreatic carcinoma cell lines kindly provided by Dr. Elizabeth Jaffee {3666}. Cell lines were cultured in DMEM supplemented with 10% FBS and antibiotics (100 units/ml penicillin and 100 ug/ml streptomycin). CAPAN1 and CAPAN2 cell lines were cultured in RPMI 1640 medium (Life Technologies, Inc, Gaithersburg, Md.) supplemented with 10% FBS and antibiotics (100 units/ml penicillin and 100 ug/ml streptomycin) respectively. Use of different media minimized the variance in growth rates that would otherwise be exaggerated with a single medium. Cells were incubated at 37° C. in a humidified atmosphere of 5% CO₂ in air.

mRNA Extractions and Affymetrix GeneChips and Data Analysis.

Sample preparation and processing procedure was performed as described in the Affymetrix GeneChip Expression Analysis Manual (Santa Clara, Calif.). Briefly, each frozen tissue was crushed to powder by using the Spex Certiprep 6800 Freezer Mill (Metuchen, N.J.). Total RNA was then extracted from the crushed normal and neoplastic tissues or cell pellets (BxPC3, COLO357, Hs766T, MiaPaCa2, Panc1, PL3, PL4, PL8) using TRIzol (Life Technologies, Inc., Rockville, Md.) and cleaned using RNeasy columns according to the manufacturer's protocol (Qiagen, Valencia, Calif.). Using 5 to 40 μg of total RNA, double-stranded cDNA was synthesized following Super-Script Choice system (Life Technologies, Inc., Rockville, Md.). T7-(dT24) oligomers was used for priming the first strand cDNA synthesis. The resultant cDNA was purified using Phase Lock Gel, phenol/chloroform extraction, and precipitated with ethanol. The cDNA pellet was collected and dissolved in appropriate volume. Using cDNA as template, cRNA was synthesized using a T7 MegaScript In Vitro Transcription (IVT) Kit (Ambion, Austin, Tex.). Biotinylate-11CTP and 16-UTP ribonucleotides (Enzo Diagnostics Inc., Farmingdale, N.Y.) were added to the reaction as labeling reagents. IVT reactions were performed at 37° C. for 6 hours and the labeled cRNA obtained was purified using RNeasy columns (Qiagen, Valencia, Calif.). The cRNA was fragmented in fragmentation buffer (40 mmol/L Tris-Acetate, pH 8.1, 100 mmol/L KOAc, 30 mmol/L MgOAc0 for 35 minutes at 94° C. Fragmented cRNA prepared from each sample (10 to 11 μg/probe array) was hybridized to the human GeneChip set (HG_U95 A, B, C, D, and E) noncompetitively at 45 C. for 24 hours in a hybridization oven with constant (60 rpm). Fragmented crNAs are hybridized to the GeneChip set by way of multiple 20 to 25 oligonucleotide probes specific for each gene, with each probe corresponding to a different region of the mRNA of interest. The probes specific for each mRNA are scattered across the surface of each GeneChip to control for technical issues that occur with each hybridization. The chips were washed and stained using Affymetrix fluidics stations. Staining was performed using streptavidin-phycoerythrin conjugate (SAPE; Molecular Probes, Eugene, Oreg.), followed by the addition of biotinylated antibody to streptavidin (Vector Laboratories,Burlingame, Calif.) and finally with streptavidin-phycoerythrin conjugate. Probe arrays were scanned using fluorometric scanners (Hewlett Packard Corporation, Palo Alto, Calif.).

The scanned images were inspected and analyzed using established quality control measures with the hybridization intensities reflecting in a linear manner the mRNA expression in the tissues or cells being assayed. Hybridization was controlled for each probe by the use of a mismatch control that has a single base mismatch. This mismatch control is analyzed using the GeneLogic informatics filter that compares the hybridization intensity of mismatched to perfect matched probes (to eliminate those that are nonspecific over a specified threshold) as well as different probes to the same gene.

Statistical Data Analysis.

The GeneExpress Software System Fold Change Analysis tool was used to identify genes expressed at least fivefold greater in the pancreatic cancers compared to normal tissues. For each gene fragment, the ratio of the geometric means of the expression intensities in the normal control tissues and the pancreas cancer samples was calculated, and the fold change then calculated on a per fragment basis. Confidence limits were calculated using a two-sided Welch modified t-test on the difference of the means of the logs of the intensities.

SAGE.

Short-term cultures of nonneoplastic pancreatic ductal epithelial cells (HX and H126) were prepared as described and validated as having the characteristics of ductal epithelium. SAGE libraries were previously constructed as described by Ryu et al (Cancer Res. 2001, 61:1833-1838; Ryu et al., Cancer Res., 2002, 62:819-826) and sequencing was performed by the CGAP SAGE consortium at the Lawrence Livermore National Laboratories and Washington University Human Genome Center (St. Louis, Mo.). SAGE library data from the short-term cultures of nonneoplastic pancreatic duct epithelial cells have been posted on the CGAP web site as part of the SAGEmap database (http://www.ncbi.nlm.nih.gov/SAGE).

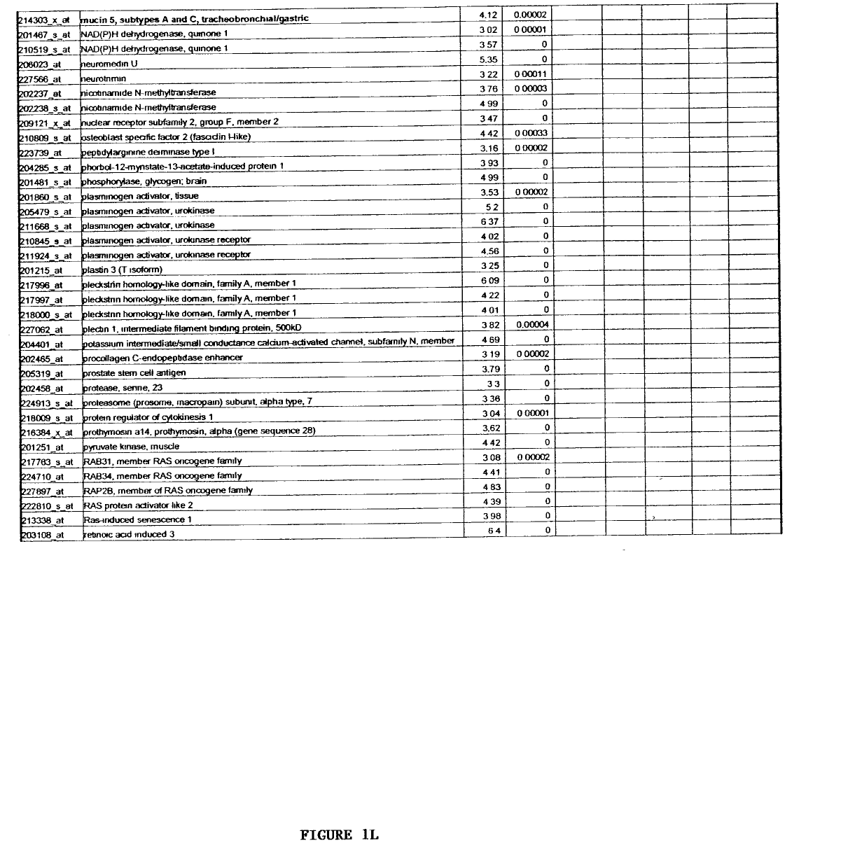

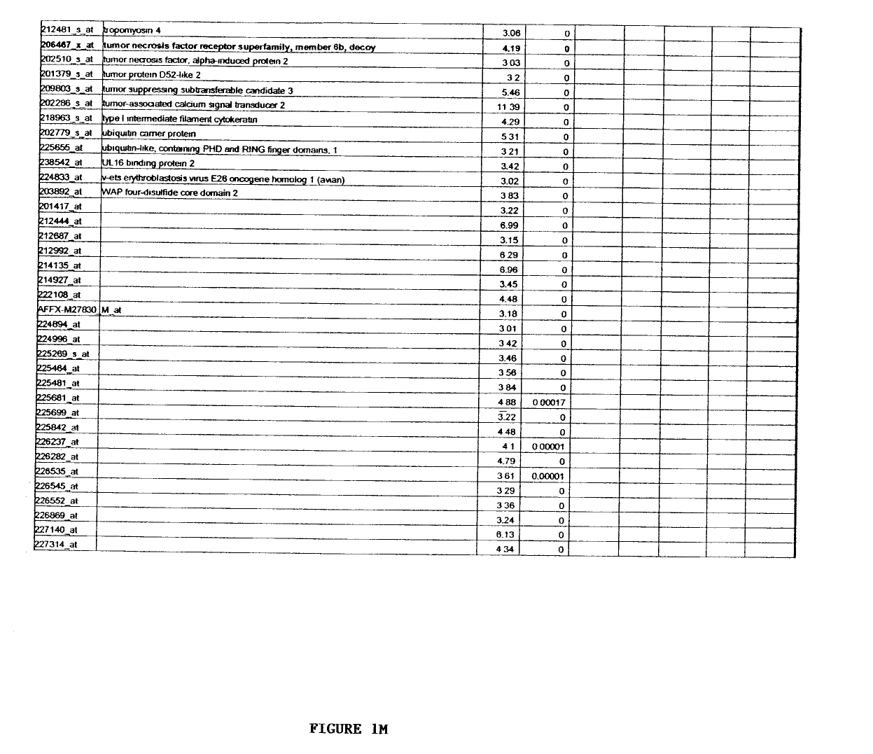

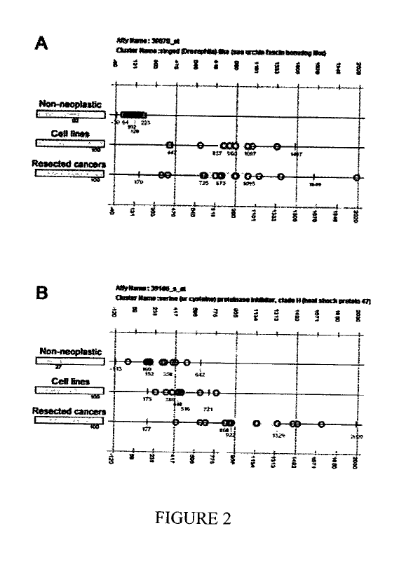

Unigene accession numbers of selected fragments are provided in FIGS. 1A through 1M. FIGS. 1A through to 1M are tabulated results showing the highly expressed genes identified in pancreatic cell lines and tissues. In FIGS. 1A to 1C, the first column identifies the nucleic acid sequence fragment name (Affymetrix gene fragments identified by the GeneExpress platform); the second column identifies the known gene name; the third column identifies the Unigene accession number; the third column identifies the fold change in gene expression as compared to normal cell gene expression; the fourth column denotes the P values; the fifth column indicates eNorthern pattern (described in FIG. 2 below); the sixth column identifies the SAGE normal tags; the seventh column indicates the novel nucleic acid molecules identified and includes previously reported genes; the eighth column provides the cellular localization of the molecules.

In FIGS. 1D to 1M, the first column identifies the nucleic acid sequence fragment name (Affymetrix gene fragments identified by the GeneExpress platform); the second column identifies the known gene name; the third column identifies the Unigene accession number; the third column identifies the fold change in gene expression as compared to normal cell gene expression; the fourth column denotes the P values.

In situ hybridization.

Preparation of digoxigenen-labeled sense and antisense riboprobes and in situ hybridization were performed as previously described in detail (Iacobuzio-Donahue et al, Am J. Pathol., 2002, 160:91-99).

RT-PCR

Total RNA was isolated from cultured cells by using TRIZOL reagent (Life Technologies, Inc.). An aliquot. of 1 μg of total RNA from each sample was reverse-transcribed to cDNA using the Superscript II kit (Life Technologies, Inc.) according to the manufacturer's instructions, with oligo(dT)₁₂₋₁₈ primer. PCR-primers were designed to amplify cDNA fragments with various sizes using standard PCR conditions. The PCR reaction products were resolved by electrophoresis in a 3% agarose gel and stained with ethidium bromide. Loading was controlled by the simultaneous PCR of glyceraldehydes-3-phosphate dehydrogenase cDNA.

Immunohistochemistry.

Sections of infiltrating primary ductal adenocarcinoma of the pancreas were formalin-fixed and paraffin-embedded, and unstained 4-μm sections were then cut from the paraffin blocks. For detection of heat shock protein 47 (hsp47), sections were deparaffinized by routine techniques before placing in 200 ml of Target Retrieval Solution, pH 6.0 (Envision Plus Detection kit, DAKO, Carpinteria, Calif.) for 20 minutes at 100° C. After cooling for 20 minutes, slides were quenched with 3% H₂O₂ for 5 minutes, before incubating with a 1:800 dilution of monoclonal antibody (colligin m16.10A1) against heat shock protein 47 (Stressgen Biotechnologies, Victoria, BC, Canada) for 30 minutes using the DAKO Autostainer. Labeling was detected with the DAKO Envision system following the manufacturer's protocol. For detection of topoisomerase IIα and fascin, slides were steamed for 20 minutes in sodium citrate buffer (diluted to 1× from 10× heat-induced epitope retrieval buffer; Ventana-Bio Tek Solutions, Tucson, Ariz.). After cooling for 5 minutes, slides were labeled with a 1:3200 dilution of mouse monoclonal antibody against topoisomerase II (clone TG100; Neomarkers, Freemont, Calif.) or a 1:500 dilution of mouse monoclonal antibody against fascin (DAKO) using the Bio Tek 1000 automated stainer (Ventana). Labeling was detected by adding biotinylated secondary antibodies, avidin-biotin complex, and 3,3′-diaminobenzidine. All sections were counterstained with hematoxylin, and staining was evaluated by three of the authors (CID, AM, and RHH) with agreement in all cases evaluated. Staining was considered positive if at least 10% of the cells showed immunolabeling.

### Example 1

### Data Filtering

Affymetrix GeneChips® were analyzed for all genes with a 5-fold or greater increase in expression in the pancreatic adenocarcinoma tumor tissues or cell lines compared to all, normal tissues. Thresholds of expression were set to include all genes that were expressed in at least 50% of the malignant samples (cell lines and tumor tissues) and in no more than 5% of the normal tissues. We identified 165 fragments expressed at least 5-fold greater in pancreatic cancer samples as compared to normal tissues, 12 of which were expressed greater than 10-fold.

### Example 2

### Identification of Highly Expressed Genes in Pancreatic Cancer

Characterization of the 165 fragments identified revealed that 56 fragments corresponded to ESTs, and 109 fragments corresponded to known genes. Among these 109 fragments, 11 genes were represented by two or more fragments, resulting in 97 known genes identified as expressed at least 5-fold greater in pancreatic cancers as compared to normal (FIGS. 1A through 1M).

The GeneExpress™ platform allows for an eNorthern™ analysis of Affymetrix fragments to determine the levels of expression of any fragment among the normal and cancer samples analyzed. An e-Northern™ was then generated for each of the 97 Affymetrix fragments to determine levels of expression of each fragment within the normal tissues, pancreas cancer cell lines, and pancreas cancer tumor tissues studied. Two prominent patterns of expression were identified (FIG. 2). The first pattern (A pattern) demonstrated elevated expression of the fragment in both pancreas cancer cell lines and in resected neoplastic pancreas tissues compared to normal tissues. X fragments showed this pattern. The second pattern (B pattern) showed elevated expression of the fragment in the resected neoplastic tissues only, but not in the cancer cell lines or normal tissues. This pattern was observed for 29 fragments.

The normal pancreas contains a predominance of acinar cells and islets relative to normal duct epithelium, the contribution of pancreatic duct epithelium. The normal pancreatic duct epithelium may therefore be underrepresented in gene expression analyses of normal pancreas. Therefore, the candidate genes identified by Affymetrix GeneChip® were further screened to exclude genes highly expressed in normal pancreatic ductal epithelial cells. For each gene identified as differentially expressed by Affymetrix GeneChip®, the corresponding SAGE tag was identified, and the total number of SAGE tags present in the SAGEmap database (http://www.ncbi.nlm.nih.gov/SAGE/) of normal pancreas duct epithelium libraries HX and H126 was determined. Any gene having more than five tags in at least one of these two SAGE libraries was then excluded from further analysis. Using this approach, 10 genes were identified as having high levels of expression in normal pancreatic duct epithelium. These genes were excluded, leaving 97 remaining differentially expressed genes.

Thus, based on the initial results of eNorthern™ analysis and SAGE filtering, 97 candidate genes were identified as potential markers of pancreatic cancer. For each of the 97 genes identified, a search was performed using the online NCBI database PubMed using the known gene name together with the terms “pancreas” or “pancreas cancer”. Of the 97 genes analyzed, 28 genes were found to be previously reported as playing a role in pancreatic cancer, whereas 69 genes were not (FIGS. 1A through 1M). Of these 69 genes not identified in this PubMed search as having been reported in pancreatic cancer, 21 have been reported before in association with tumor types other than pancreatic cancer, while 48 genes have never been reported in association with any neoplasm.

These 97 candidate tumor markers of pancreatic cancer represented a variety of cellular functions. Genes identified included those involved in cell membrane junctions (claudin 1, connexin 26), signal transduction (tumor associated calcium signal transducer 2, ras GTPase activating protein-like), calcium homeostasis (S100 calcium binding protein P), cytoskeletal assembly (fascin, keratin 7 and rabkinesin6), cell surface adhesion and recognition (integrin beta-like 1), DNA transcription (topoisomerase 11 alpha, transcription factor BMAL2 and AML1), DNA repair (pleckstrin, ATDC), or extracellular matrix remodeling and function (heat shock protein 47, MMP14, and MMP7). The cellular localization of the corresponding gene products was also determined. Genes were found to encode membrane bound proteins (prostate stem cell antigen, OB-cadherin), cytoplasmic proteins (fascin, ATDC), nuclear proteins (topoisomerase 11 alpha, pleckstrin, paraneoplastic antigen MA1), as well as extracellular proteins, such as those involved in extracellular matrix homeostasis (heat shock protein 47, thrombospondin 2) or secreted protein products (osteopontin).

### Example 3

### Verification of Selected Candidate Tumor Markers

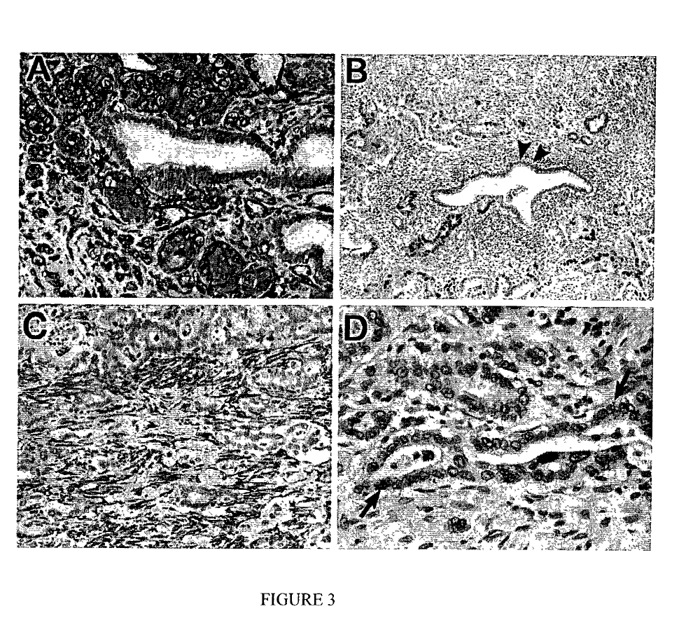

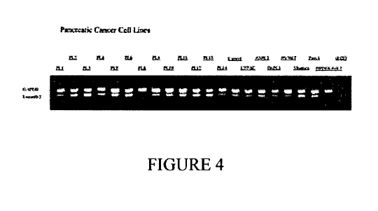

Candidate genes were selected for verification of expression in samples of pancreatic cancer tissues or cell lines (FIGS. 3 and 4). Four genes were selected for immunchistochemical or in situ hybridization labeling: fascin, topoisomerase II alpha, heat shock protein 47 (hsp47), and pleckstrin.

Fascin and topoisomerase II both showed an “A” pattern of expression on eNorthern™, corresponding to elevated expression in both the resected neoplastic tissues and cancer cell lines. Immunohistochemical labeling of fascin showed intensely positive membranous staining of the neoplastic epithelium in eight of eight samples of paraffin-embedded pancreatic duct adenocarcinomas studied (100%). In all cases, normal duct epithelium did not express fascin protein (FIG. 3, panel A).

Topoisomerase II alpha also showed strong positive nuclear immunolabeling within eight of eight pancreatic duct adenocarcinomas studied (100%). Normal duct epithelium and the surrounding desmoplastic stroma were negative for expression of topoisomerase II alpha. (FIG. 3, panel B).

In contrast to fascin and topoisomerase II alpha, hsp47 showed a “B” pattern of expression on eNorthern™, indicating elevated expression of hsp47 in the resected neoplastic tissues only, but not in the cancer cell lines or normal tissues. (FIG. 3, panel C). In concordance with this pattern, immunolabeling for hsp47 showed strong cytoplasmic labeling of the desmoplastic stroma within the invasive cancer in eight of eight pancreatic duct adenocarcinomas studied (100%). In one of the eight cases, the neoplastic epithelium also labeled. Furthermore, no expression of hsp47 was observed within normal pancreatic duct epithelium, nor within the intralobular stroma of normal pancreas tissue present within the same paraffin-embedded tissue sections.

Pleckstrin was also identified as differentially expressed in pancreatic cancer and it had an “A” pattern of expression by eNorthern™. No commercially available antibody for pleckstrin was available. Therefore, a digoxigenen-labeled probe was generated to match the coding region of the pleckstrin gene for use in in situ hybridization. In situ hybridization using the antisense probe showed expression within the neoplastic epithelium in eight of eight cases, seen as variably sized granules throughout the cytoplasm of the neoplastic epithelium, in contrast to normal duct epithelium or the surrounding desmoplastic stroma which did not express this gene (FIG. 3, panel D).

Eight additional genes were selected for validation by RT-PCR study of 20 pancreas cancer cell lines (FIG. 4). Genes selected for validation using RT-PCR were claudin 1, S100 calcium-binding protein P (S100P), interferon induced transmembrane protein 1 (IFITM1), lamin B2, DKFZP564G013 protein, KIAA0470 gene product, KIAA1265 protein, and KIAA1363 protein. All genes showed detectable expression within 18 of 20 cell lines analyzed, in support of their initial identification as highly expressed genes by Affymetrix GeneChip®.

The foregoing description of the invention is merely illustrative thereof, and it is understood that variations and modifications can be made without departing from the scope of spirit of the invention as set forth in the following claims.

What is claimed is:

1. A method for detection of cancer, comprising detecting one or more nucleic acid molecules in a subject sample, wherein at least one of the molecules comprises a sequence corresponding to a molecule identified in any of FIGS. 1A through 1M.

2. The method of claim 1 wherein the presence of the one or more nucleic acid molecules is correlated to a sample of a normal subject.

3. The method of claim 1 wherein the sample is obtained from a mammal suspected of having a proliferative cell growth disorder.

4. The method of claim 1 wherein the sample is obtained from a mammal suspected of having a pancreatic cancer.

5. The method of claim 1, wherein at least one of the molecules comprises a sequence corresponding to SEQ ID NOs: 1 through 87.

6. The method of any one of claims 1 through 5, wherein the nucleic acid molecule comprises a sequence having at least about 80% sequence identity to a molecule identified in any of FIGS. 1A through 1M.

7. The method of any one of claims 1 through 5, wherein the nucleic acid molecule comprises a sequence having at least about 90% sequence identity to a molecule identified in any of FIGS. 1A through 1M.

8. The method of any one of claims 1 through 5, wherein the nucleic acid molecule comprises a sequence having at least about 95% sequence identity to a molecule identified in any of FIGS. 1A through 1M.

9. The method of any one of claims 1 through 8, wherein the nucleic acid molecule is expressed at least a higher level in a patient with cancer as compared to expression levels in a normal individual.

10. The method of any one of claims 1 through 8, wherein the nucleic acid molecule is expressed at least about 5 fold higher in a patient with cancer as compared to expression in a normal individual.

11. The method of any one of claims 1 through 8, wherein the nucleic acid molecule is expressed at least about 10 fold higher in a patient with cancer as compared to expression in a normal individual.

12. The method of any one of claims 8 through 11 wherein the cancer is a pancreatic cancer.

13. The method of any one of claims 1 through 12 wherein the nucleic acid molecule encodes for a membrane bound protein, cytoplasmic protein, nuclear protein or extracellular protein.

14. The method of claim 13 wherein the protein comprises OB-cadherin, fascin, ATDC, topoisomerase II alpha, pleckstrin, paraneoplastic antigen MA1, heat shock protein 47, thrombospondin 2, or osteopontin.

15. The method of any one of claims 1 through 14 wherein the subject sample is obtained from a mammalian patient.

16. The method of any one of claims 1 through 14 wherein the subject sample is obtained from a human patient.

17. The method of any one of claims 1 through 16 wherein a polypeptide encoded by the nucleic acid molecule is detected.

18 A method for detection of cancer, comprising detecting in a subject sample a polypeptide encoded by a sequence comprising a sequence identified in any of FIGS. 1A through 1M.

19. The method of claim 18 wherein a polypeptide encoded by a sequence comprising a sequence identified in any of SEQ ID NOs: 1 through 87.

20. The method of claims 18 or 19 wherein the polypeptide is encoded by a sequence having at least about 80% sequence identity to a molecule identified in any of FIGS. 1A through 1M.

21. The method of claim 18 or 19 wherein the polypeptide is encoded by a sequence having at least about 90% sequence identity to a molecule identified in any of FIGS. 1A through 1M.

22. The method of claim 18 or 19 wherein the polypeptide is encoded by a sequence having at least about 95% sequence identity to a molecule identified in any of FIGS. 1A through 1M.

23. The method of any one of claims 18 through 22 wherein the polypeptide is expressed at least a higher level in a patient with cancer as compared to expression levels in normal individuals.

24. The method of any one of claims 18 through 22 wherein the polypeptide is expressed at least about 5 fold higher in a patient with cancer as compared to expression in a normal individual.

25. The method of any one claims 18 through 22 wherein the polypeptide is expressed at least about 10 fold higher in a patient with cancer as compared to expression in a normal individual.

26. The method of any one of claims 22 through 25 wherein the cancer is a pancreatic cancer.

27. The method of any one of claims 18 through 26 wherein the subject sample is obtained from a mammalian patient.

28. The method of any one of claims 18 through 26 wherein the subject sample is obtained from a human patient.

29. A method for identifying a candidate therapeutic agent comprising:
contacting a candidate agent with a nucleic acid molecule or expression product thereof where the nucleic acid molecule comprises a sequence identified in any of FIGS. 1A through 1M; and
detecting interaction between the candidate agent and the nucleic acid molecule or expression product.

30. The method of claim 29 wherein the nucleic acid molecule comprises a sequence identified in any of SEQ ID NOs: 1 through 87.

31. The method of claims 29 or 30 wherein the nucleic acid molecule comprises a sequence having at least about 80% sequence identity to a sequence identified in any of FIGS. 1A through 1M.

32. The method of claims 29 or 30 wherein the nucleic acid molecule comprises a sequence having at least about 90% sequence identity to a sequence identified in any of FIGS. 1A through 1M.

31. The method of claims 29 or 30 wherein the nucleic acid molecule comprises a sequence having at least about 95% sequence identity to a sequence identified in any of FIGS. 1A through 1M.

32. A method for identifying a candidate therapeutic agent comprising:
contacting a candidate agent with a nucleic acid molecule or expression product thereof where the nucleic acid molecule or expression product are overexpressed in a mammal suffering from a pancreatic cancer; and
detecting interaction between the candidate agent and the nucleic acid molecule or expression product.

33. The method of any one of claims 29 through 32 wherein the candidate compound is selected from the group consisting of a protein, a peptide, an oligopeptide, a nucleic acid, a small organic molecule, a polysaccharide and a polynucleotide.

34. The method of any one of claims 29 through 32 wherein the nucleic acid molecule or expression product are provided on a solid support.

35. The method of any one of claims 29 through 34 wherein binding of the candidate compound is detected.

36. A method for treating a mammal suffering from cancer comprising administering to the mammal an antibody or lymphocyte specific for a nucleic acid molecule or expression product thereof where the nucleic acid molecule comprises a sequence corresponding to a molecule identified in any of FIGS. 1A through 1M.

37. The method of claim 36 wherein the nucleic acid molecule comprises a sequence identified in any of SEQ ID NOs: 1 through 87.

38. The method of claims 36 or 37 wherein the nucleic acid molecule comprises a sequence having at least about 80% sequence identity to a sequence identified in any of FIGS. 1A through 1M.

39. The method of claims 36 or 37 wherein the nucleic acid molecule comprises a sequence having at least about 90% sequence identity to a sequence identified in any of FIGS. 1A through 1M.

40. The method of claims 36 or 37 wherein the nucleic acid molecule comprises a sequence having at least about 95% sequence identity to a sequence identified in any of FIGS. 1A through 1M.

41. The method of any one of claims 36 through 40 wherein the patient is suffering from a pancreatic cancer.

42. A pharmaceutical composition comprising an antibody or lymphocyte specific for a nucleic acid molecule or expression product thereof where the nucleic acid molecule comprises a sequence corresponding to a molecule identified in any of FIGS. 1A through 1M.

43. The pharmaceutical composition of claim 40 wherein the nucleic, acid molecule comprises a sequence having sequence identity to a molecule identified in any of SEQ ID NOs: 1 through 87.

44. The pharmaceutical composition of claims 42 or 43 wherein the nucleic acid molecule comprises a sequence having at least about 80% sequence identity to a molecule identified in any of FIGS. 1A through 1M.

45. The pharmaceutical composition of claims 42 or 43 wherein the nucleic acid molecule comprises a sequence having at least about 90% sequence identity to a molecule identified in any of FIGS. 1A through 1M.

46. The pharmaceutical composition of claims 42 or 43 wherein the nucleic acid molecule comprises a sequence having at least about 95% sequence identity to a molecule identified in any of FIGS. 1A through 1M.

47. The pharmaceutical composition of any one of claims 42 through 46 wherein the antibody or lymphocyte are packaged together with written instructions for treatment of cancer.

48. The pharmaceutical composition of any one of claims 42 through 46 wherein the antibody or lymphocyte are packaged together with written instructions for treatment of pancreatic cancer.

49. An antibody or lymphocyte specific for a nucleic acid molecule or expression product thereof where the nucleic acid molecule comprises a sequence corresponding to a molecule identified in any of FIGS. 1A through 1M.

50. The antibody or lymphocyte of claim 49 wherein the nucleic acid molecule comprises a sequence having a sequence identity to a molecule identified in any of SEQ ID NOs 1 through 87.

51. The antibody or lymphocyte of claims 49 or 50 wherein the nucleic acid molecule comprises a sequence having at least about 80% sequence identity to a molecule identified in any of FIGS. 1A through 1M.

52. The antibody or lymphocyte of claims 49 or 50 wherein the nucleic acid molecule comprises a sequence having at least about 90% sequence identity to a molecule identified in any of FIGS. 1A through 1M.

53. The antibody or lymphocyte of claims 49 or 50 wherein the nucleic acid molecule comprises a sequence having at least about 95% sequence identity to a molecule identified in any of FIGS. 1A through 1M.

54. A diagnostic kit comprising a reaction body and a molecule substantially complementary to a sequence corresponding to a molecule identified in any of FIGS. 1A through 1M.

55. The diagnostic kit comprising a reaction body and a molecule substantially complementary to a sequence corresponding to a molecule identified in any of SEQ ID NOs 1 through 87.

56. The kit of claims 54 or 55 wherein the sequence has at least about 80% sequence identity to a molecule identified in any of FIGS. 1A through 1M.

57. The kit of claims 54 or 55 wherein the sequence has at least about 90% sequence identity to a molecule identified in any of FIGS. 1A through 1M.

58. The kit of claims 54 or 55 wherein the sequence has at least about 95% sequence identity to a molecule identified in any of FIGS. 1A through 1M.

59. A method for treating a mammal with cancer, comprising:
administering to the mammal an effective amount of a nucleic acid sequence wherein the nucleic acid molecule comprises a sequence corresponding to a molecule identified in any of FIGS. 1A through 1M.

60. The method of claim 59 wherein the nucleic acid molecule comprises a sequence corresponding to a molecule identified in any of SEQ ID NOs 1 through 87.

61. The method of claim 59, wherein the mammal is a human.

62. The method of claims 59 or 60, wherein the sequence has at least about 80% sequence identity to a molecule identified in any of FIGS. 1A through 1M.

61. The method of claims 59 or 60, wherein the sequence has at least about 90% sequence identity to a molecule identified in any of FIGS. 1A through 1M.

62. The method of claims 59 or 60, wherein the sequence has at least about 95% sequence identity to a molecule identified in any of FIGS. 1A through 1M.

63. The method of claims 59 or 60, wherein the cancer is pancreatic cancer.

64. A vector comprising one or more nucleic acid molecules, or variants thereof, where the nucleic acid molecule comprises a sequence corresponding to a molecule identified in any of FIGS. 1A through 1M.

65. The vector of claim 64, wherein the nucleic acid molecule comprises a sequence corresponding to a molecule identified in any of SEQ ID NOs 1 through 87.

66. The vector of claims 64 or 65, wherein the sequence has at least about 80% sequence identity to a molecule identified in any of FIGS. 1A through 1M.

67. The vector of claims 64 or 65, wherein the sequence has at least about 90% sequence identity to a molecule identified in any of FIGS. 1A through 1M.

68. The vector of claims 64 or 65, wherein the sequence has at least about 95% sequence identity to a molecule identified in any of FIGS. 1A through 1M.
